(19) United States
(12) Patent Application Publication
(10) Pub. No.: US 2026/0218106 A1
(43) Pub. Date: Jul. 30, 2026

(54) FERMENTATION DEVICE FOR RECOVERING CARBOXYLIC ACIDS WHILE REDUCING GREENHOUSE GAS EMISSIONS

(71) Applicants: Shanghai Municipal Engineering Design Institute (Group) Co., Ltd., Shanghai (CN); Tongji University, Shanghai (CN); Wuxi Xinjiayuan Environmental Protection Co., Ltd, Yixing (CN)

(72) Inventors: Lei Dong, Shanghai (CN); Xin Zhang, Shanghai (CN); Yinguang Chen, Shanghai (CN); Chao Liu, Shanghai (CN); Jiaping Yang, Yixing (CN); Guoqiang Wang, Yixing (CN); Yubo Li, Shanghai (CN); Yuwei She, Shanghai (CN)

(21) Appl. No.: 19/445,477

(22) Filed: Jan. 10, 2026

(30) Foreign Application Priority Data
Jan. 24, 2025 (CN) 2025101190976

(51) Int. Cl. C12M 1/06; C12M 1/26; C12M 1/36

(52) U.S. Cl. CPC C12M 27/08; C12M 33/04; C12M 41/48

(57) ABSTRACT

A fermentation device for recovering carboxylic acids while reducing greenhouse gas emissions includes a fermentation container, a sludge inlet assembly, a decanting assembly, a hydraulic circulation assembly, a heating and insulation assembly, an agitator assembly, a reagent regulation assembly, and a monitoring and electrical control assembly. The fermentation device ferments all primary and excess sludge from a sewage treatment plant to recover the carboxylic acids. Through operation of the fermentation device and control of reaction parameters, a carboxylic acid mixture with a specific ratio is produced, thereby significantly reducing greenhouse gases such as methane produced during fermentation. The carboxylic acid mixture recovered is added to a biological reaction tank to achieve clean ultra-purification treatment of sewage and synergistic and efficient removal of the greenhouse gases. The carboxylic acid mixture can also be purified by a separating supernatant to recover carboxylic acid industrial products for resource recovery.

### CROSS-REFERENCE TO RELATED APPLICATION

This application claims priority to Chinese Patent Application No. 202510119097.6, filed on Jan. 24, 2025, which is herein incorporated by reference in its entirety.

### TECHNICAL FIELD

The disclosure relates to the technical field of municipal drainage, and more particularly to a fermentation device and a fermentation method for recovering carboxylic acids while reducing greenhouse gas emissions.

### BACKGROUND

Currently, many sewage treatment plants add large amounts of additional carbon sources, such as methanol, acetic acid, sodium acetate, propionic acid and glucose, due to insufficient carbon sources required for denitrification and phosphorus removal in sewage treatment. In recent years, with continuous improvement of requirements for effluent water quality across regions, demand for the carbon sources has gradually increased, leading to a doubling of energy consumption in sewage treatment plants. Additionally, disposal of large amounts of sludge generated during a sewage treatment process is difficult, and an issue of resource utilization of the sludge urgently needs to be addressed.

Latest research shows that the sludge generated by the sewage treatment plants (mainly including sludge from a pre-treatment stage and excessive sludge from a biological treatment stage) contains a large amount of carbohydrates, proteins, and fats, all of which belong to slow-release organic carbon sources. When converted into efficient organic carbon sources (such as carboxylic acids, including acetic acid, propionic acid, butyric acid, and valeric acid), these slow-release organic carbon sources can be used to significantly promote denitrification and phosphorus removal processes in biological reaction tanks. By adjusting a potential of hydrogen (pH) value of the sewage and concentrations of trace elements in the sewage, metabolic balance of functional microorganisms in the biological reaction tanks can be regulated, thereby inhibiting metabolic activity of non-functional microorganisms (such as methanogenic bacteria), avoiding emissions of greenhouse gases such as methane, and achieving a goal of carbon reduction. Therefore, utilizing the sludge to prepare the efficient organic carbon sources of the carboxylic acids is particularly important for promoting low-carbon operation of the sewage treatment plants.

Currently, research in the field of sludge fermentation at home and abroad mainly focuses on mechanisms of acid production under different fermentation conditions of the sludge, emphasizing theoretical analysis. However, a progress of device development in practical engineering applications is relatively slow. In an anaerobic fermentation process of the sludge, requirements of bacterial strains for the pH value, temperature, etc. of the sludge are relatively complex. In practical large-scale engineering applications, most devices in the art are limited to adding reagents into the sludge for pretreatment or only consider device configurations to meet fermentation conditions, resulting in high costs for reagent addition and temperature control. For example, a Chinese patent with an application No. 202211043645.4 and a publication No. CN116022985B discloses a preparation method for carbon source from primary sludge in a sewage treatment plant, pretreating the sludge by adjusting the pH value to an equipotential point. A Chinese patent with an application No. 202321878183.8 and a publication No. CN220393692U discloses a device for producing acid by anaerobic fermentation of excess sludge based on potassium ferrate, pretreating the sludge by adding the reagents. These technologies have weakened process control of sludge fermentation itself and have not yet been widely applied in engineering.

To sum up, the technical field of municipal drainage lacks a fermentation device that integrates hydraulic condition optimization and substrate metabolism regulation, enabling efficient recovery of the carboxylic acids at low cost while reducing greenhouse gas emissions.

### SUMMARY

An objective of the disclosure is to improve internal hydraulic conditions within a sludge anaerobic fermentation device, regulate substrate metabolism, and optimize process parameters, thereby enabling more efficient, energy-saving, and low-carbon fermentation of sludge generated by sewage treatment plants within a fermentation device to produce high-quality carbon sources. These high-quality carbon sources are then recycled back to biological reaction tanks of the sewage treatment plants, significantly improving denitrification and phosphorus removal efficiency in the biological reaction tanks, reducing an amount of externally added carbon sources required by the biological reaction tanks, and cutting greenhouse gas emissions, thereby achieving a goal of cost reduction and improved efficiency.

To achieve the aforementioned objective, a fermentation device for recovering carboxylic acids while reducing greenhouse gas emissions is provided by the disclosure. The fermentation device for recovering the carboxylic acids while reducing the greenhouse gas emissions includes a fermentation container, a sludge inlet assembly, a decanting assembly, a hydraulic circulation assembly, a heating and insulation assembly, an agitator assembly, a reagent regulation assembly, and a monitoring and electrical control assembly. A sludge inlet and a hydraulic circulation inlet are defined on a lower part of a side wall of the fermentation container, a hydraulic circulation outlet is defined on a middle-upper part of the side wall of the fermentation container, and a carboxylic acid outlet is defined on an upper part of the side wall of the fermentation container. The sludge inlet assembly is connected to the sludge inlet and extends into an interior of the fermentation container. The decanting assembly is disposed inside a top of the fermentation container, and the decanting assembly is connected to the carboxylic acid outlet to decant the carboxylic acids fermented from the top of the fermentation container and transport the carboxylic acids fermented to a biological reaction tank. The hydraulic circulation assembly is connected to the hydraulic circulation inlet and the hydraulic circulation outlet, and the hydraulic circulation assembly is configured to agitate the sludge inside the fermentation container by suction and recirculation of the sludge through a circulation pump. The heating and insulation assembly is disposed on the side wall of the fermentation container and is configured to heat and insulate the sludge inside the fermentation container. The agitator assembly is configured to fully stir the sludge through multi-stage blades disposed inside the fermentation container. The reagent regulation assembly is connected to the fermentation container and is configured to deliver reagents into the fermentation container. The monitoring and electrical control assembly includes a controller and monitoring instruments disposed on the fermentation container. The monitoring instruments are configured to continuously monitor a temperature, a pH value, and water quality of the sludge inside the fermentation container to obtain monitoring results. The controller is configured to regulate process parameters of the sludge inlet assembly, the hydraulic circulation assembly, the heating and insulation assembly, the agitator assembly, and the reagent regulation assembly in real time based on the monitoring results to ensure that the temperature of the sludge inside the fermentation container (1) is stably maintained in a range of 35 degrees Celsius (° C.) to 55° C., thereby producing the carboxylic acids of a predetermined concentration. A calculation formula for a concentration Sᵥ of the carboxylic acids is expressed as follows:

$$\begin{matrix}{S}_{v} = \int [ \frac{{N}_{1} + {aN}_{2}}{{N}_{1} + {N}_{2}} \cdot ( \frac{{k}_{m , h} \cdot {X}_{h}}{1 + \frac{{K}_{S , h}}{{S}_{h}} + \frac{{S}_{v}}{{K}_{I , h}}} - \frac{{k}_{m , v} \cdot {X}_{v}}{1 + \frac{{K}_{S , h}}{{S}_{h}} + \frac{{S}_{v}}{{K}_{I , h}}} ) ] dt \\ {S}_{h} = \int [ \frac{{N}_{1} + {aN}_{2}}{{N}_{1} + {N}_{2}} \cdot ( k \cdot {S}_{P} - \frac{{k}_{m , h} \cdot {X}_{h}}{1 + \frac{{K}_{S , h}}{{S}_{h}} + \frac{{S}_{v}}{{K}_{I , h}}} ) ] dt \\ {S}_{P} = \int [ \frac{{N}_{1} + {aN}_{2}}{{N}_{1} + {N}_{2}} \cdot ( - k \cdot {S}_{P} + {k}_{d , h} \cdot {X}_{h} + {k}_{d , v} \cdot {X}_{v} ) ] dt\end{matrix}$$

where Sᵥ represents the concentration of the carboxylic acids, measured as chemical oxygen demand (COD); Sₚ represents a concentration of organic matter in sludge to-be-treated, measured as COD; Sₕ represents a concentration of reaction hydrolysis products, including concentrations of protein, fat, and carbohydrate, measured as COD; N₁ represents a variable frequency output power of the circulation pump for hydraulic stirring; N₂ represents a variable frequency output power of an agitator for mechanical stirring; a represents a regulation coefficient of stirring intensity of the agitator; k_{m,h} represents a maximum specific utilization rate of the reaction hydrolysis products; Xₕ represents a microbial concentration of carboxylic acid-producing bacteria; K_{S,h} represents a half-saturation constant for growth of the carboxylic acid-producing bacteria; K_{I,h} represents a influence coefficient of the concentration of the carboxylic acids on the growth of the carboxylic acid-producing bacteria; k_{m,v} represents a maximum specific utilization rate of the carboxylic acids; Xᵥ represents a microbial concentration of methanogenic bacteria; k represents a maximum specific substrate removal rate constant; k_{d,h} represents a microbial endogenous decay rate of the carboxylic acid-producing bacteria; and k_{d,v} represents a microbial endogenous decay rate of the methanogenic bacteria.

In an embodiment, the sludge inlet assembly includes a sludge inlet pipe, a sludge inlet pump with an adjustable flow rate, and a sludge inlet pipe bracket. The sludge inlet pipe penetrates through the sludge inlet on the side wall of the fermentation container and extends into the fermentation container until a sludge inlet pipe outlet is located in a central area of the fermentation container. The sludge inlet pipe bracket is supported below the sludge inlet pipe. The sludge inlet pump is connected to an inlet of the sludge inlet pipe and is configured to pump primary sludge or excess sludge generated by an external sewage treatment facility. Specifically, the external sewage treatment facility includes a primary sedimentation tank, a secondary sedimentation tank, and the biological reaction tank. The monitoring and electrical control assembly is configured to regulate the flow rate of the sludge inlet pump of the sludge inlet assembly in real time based on the monitoring results and control a fermentation residence duration of the sludge inside the fermentation container in a range of 5 days (d) to 8 d, thereby making a sludge inlet flow rate match a production efficiency of the carboxylic acids.

In an embodiment, a scum discharge port is defined on the upper part of the side wall of the fermentation container. The carboxylic acid outlet is connected to the biological reaction tank of the external sewage treatment facility to transport clarified liquid of the carboxylic acids fermented to the biological reaction tank. The decanting assembly includes a decanter, a carboxylic acid pipeline, a scum skimmer, and a scum collection tank. The decanter includes a decanting tank in a rectangular or annular shape, and a tank opening of the decanting tank is defined inside the top of the fermentation container. The carboxylic acids generated by fermentation of the sludge clarify and stratify at a top of the sludge, and the clarified liquid of the carboxylic acids at the top of the sludge flows into the decanting tank through the tank opening of the decanting tank in an overflow manner. An end of the carboxylic acid pipeline is connected to a tank body of the decanting tank, and another end of the carboxylic acid pipeline is connected to the carboxylic acid outlet. The scum skimmer is disposed on the top of the fermentation container, is driven by a scum skimmer motor, and is configured to skim scum on a liquid surface of the clarified liquid of the carboxylic acids into the scum collection tank. A tank opening of the scum collection tank is 0.5 centimeters (cm) to 5 cm higher than the liquid surface of the clarified liquid of the carboxylic acids, a tank body of the scum collection tank is inclined downward, and a lowest part of the scum collection tank is connected to the scum discharge port. The scum collected is discharged from the fermentation container through the scum discharge port.

In an embodiment, the hydraulic circulation assembly includes the circulation pump, a circulation sludge inlet main pipe, a circulation sludge inlet distribution head, circulation sludge inlet branch pipes, and a circulation sludge outlet pipe. The circulation sludge inlet distribution head is disposed in the central area of a bottom of the fermentation container, and the circulation sludge inlet branch pipes are uniformly connected around the circulation sludge inlet distribution head in a radial pattern. An end of the circulation sludge inlet main pipe is connected to the circulation sludge inlet distribution head, and another end of the circulation sludge inlet main pipe extends outward through the hydraulic circulation inlet. The circulation pump is disposed outside the fermentation container, an inlet of the circulation pump is connected to an outlet of the circulation sludge inlet main pipe, and an outlet of the circulation pump is connected to the circulation sludge outlet pipe located at a middle-upper part of the fermentation container. The circulation sludge outlet pipe extends into the fermentation container through the hydraulic circulation outlet, and multiple circulation sludge outlets are defined on the circulation sludge outlet pipe. During work, the circulation pump draws the sludge from the bottom of the fermentation container through the circulation sludge inlet branch pipes and the circulation sludge inlet main pipe, then pressurizes and pumps the sludge to the circulation sludge outlet pipe, and sprays the sludge at high speed through the circulation sludge outlet. A working flow rate of the circulation pump is taken as 10-50 times a value of “an effective volume of the fermentation container ÷24”, thereby achieving hydraulic circulation of the sludge within the fermentation container and optimizing a hydraulic flow state of the sludge.

In an embodiment, the heating and insulation assembly includes a heating component and an insulation layer. The heating component is an electric heat tracing cable or a hot water coil heated by a water source heat pump wrapped around at least one of the sludge inlet pipe of the sludge inlet assembly and the side wall of the fermentation container, or the heating component is a heating structure disposed inside the fermentation container. The insulation layer is disposed on the side wall of the fermentation container and is configured to reduce a heat loss of the fermentation container. The monitoring and electrical control assembly is configured to regulate the heating and insulation assembly in real time to maintain the temperature of the sludge inside the fermentation container stable in a range of 35° C. to 55° C.

In an embodiment, a top surface of the fermentation container defines an agitator installation hole. The agitator assembly includes the agitator with a variable frequency adjustable speed, the multi-stage blades, a rotating shaft, and a heat transfer circulation pipeline. The agitator is eccentrically disposed in the agitator installation hole, and the rotating shaft extends into the fermentation container. The multi-stage blades are disposed on a middle-lower part of the rotating shaft and are evenly divided into 2-5 layers, and the multi-stage blades are configured to fully agitate the sludge inside the fermentation container. A rotating speed of the multi-stage blades is 20 revolutions per minute (rpm) to 60 rpm. Each layer of the multi-stage blades includes multiple blade plates, hydraulic support rods, and a flexible connector. The multiple blade plates are connected to each other by the flexible connector, and angles among the multiple blade plates are adjusted by expansion and contraction of the hydraulic support rods. When a larger agitation intensity of the sludge is required, the angles among the multiple blade plates are increased, and when a smaller agitation intensity of the sludge is required, the angles among the multiple blade plates are decreased.

In an embodiment, the heat transfer circulation pipeline penetrates through the rotating shaft and the multiple blade plates. The heat transfer circulation pipeline includes a heat inlet pipe and a heat return pipe connected to the heat inlet pipe. The heat inlet pipe is configured to transport a heat transfer medium supplied by the water source heat pump to thereby conduct heat to the sludge through the rotating shaft and the multiple blade plates. The heat return pipe is configured to transport cooling water back to the water source heat pump.

In an embodiment, the reagent regulation assembly includes a reagent distributor, a reagent delivery pipe provided with a metering pump, and a reagent box. The reagent box is disposed outside the fermentation container and is connected to the reagent distributor disposed inside the fermentation container through the reagent delivery pipe penetrating through the fermentation container. The reagents in the reagent box include one or more selected from the group consisting of alkaline solution, acidic solution, and trace element solution. The monitoring and electrical control assembly is configured to regulate flow rates of the alkali solution, the acid solution, and the trace element solution in the reagent regulation assembly in real time through the metering pump, to thereby maintain the pH value of the sludge inside the fermentation container at 8-10 and maintain concentrations of trace elements in the sludge as follows: copper ion (Cu²⁺) in a range of 20 millimoles per liter (mmol/L) to 65 mmol/L, manganese ion (Mn²⁺) in a range of 2.5 mmol/L to 6.0 mmol/L, boron ion (B³⁺) in a range of 15.0 mmol/L to 28.5 mmol/L, molybdenum ion (Mo⁶⁺) in a range of 4.0 mmol/L to 6.2 mmol/L, wolframium ion (W⁶⁺) in a range of 7.0 mmol/L to 12.2 mmol/L, nickel ion (Ni²⁺) in a range of 3.0 mmol/L to 5.5 mmol/L, cobalt ion (Co²⁺) in a range of 10.0 mmol/L to 18.3 mmol/L, zinc ion (Zn²⁺) in a range of 25.5 mmol/L to 35.0 mmol/L, and calcium ion (Ca²⁺) in a range of 10.5 mmol/L to 30.5 mmol/L.

In an embodiment, the reagent distributor is an annular reagent distributor. The annular reagent distributor is disposed 20-100 cm below the liquid surface in the fermentation container. Multiple reagent nozzles are defined on the annular reagent distributor and configured to uniformly spray the reagents into the sludge.

In an embodiment, the reagent regulation assembly further includes an inert gas storage chamber, an inert gas delivery pipe, and multiple inert gas blowing nozzles. The inert gas storage chamber is disposed inside the reagent box and is connected to the inert gas delivery pipe. An end portion of the inert gas delivery pipe is horizontally disposed at the bottom of the fermentation container. The multiple inert gas blowing nozzles are evenly distributed on the end portion of the inert gas delivery pipe. The multiple inert gas blowing nozzles are configured to spray nitrogen, helium, or argon at a predetermined flow rate to blow off residual dissolved oxygen in the sludge, thereby maintaining an anaerobic environment for fermentation of the sludge.

In an embodiment, the fermentation device for recovering the carboxylic acids while reducing the greenhouse gas emissions further includes a venting assembly. A venting port is defined on the bottom of the fermentation container; the venting assembly includes multiple venting branch pipes, a venting intermediate box, and a venting pipe. An inlet of each of the multiple venting branch pipes faces downward, with a distance of 3-30 cm from a bottom surface of the fermentation container. The venting intermediate box is a hollow box body, and a side wall of the venting intermediate box is connected to the multiple venting branch pipes. An end of the venting pipe is connected to the venting intermediate box, and another end of the venting pipe penetrates through the fermentation container through the venting port. The venting pipe is configured to completely discharge the sludge and the carboxylic acids inside the fermentation container during maintenance for the fermentation device.

In an embodiment, multiple instrument monitoring ports are defined on the side wall of the fermentation container, and the multiple instrument monitoring ports are uniformly distributed along the side wall of the fermentation container at a certain rotation angle from high to low. The monitoring instruments are embedded in the multiple instrument monitoring ports, and the monitoring instruments include thermometers, pH meters, and water quality monitoring probes. The thermometers, the pH meters, and the water quality monitoring probes are respectively used to continuously monitor the temperature, the pH value, and the water quality of the sludge inside the fermentation container. The water quality monitoring probes can simultaneously monitor the concentrations of organic matter (measured as COD), total nitrogen (TN), total phosphorus (TP), total organic acid anions, acetate, propionate, butyrate, and valerate in the sludge. The controller is configured to regulate a flow rate of the circulation pump in the hydraulic circulation assembly and a rotating speed of the agitator in the agitator assembly in real time, to thereby make data monitored by the thermometers at different positions tend to be consistent, data monitored by the pH meters at different positions tend to be consistent, and data monitored by the water quality monitoring probes at different positions tend to be consistent.

Compared with the related art, the fermentation device for recovering the carboxylic acids while reducing the greenhouse gas emissions provided by the disclosure has at least the following beneficial effects.

### 1. High Degree of Automation and Real-Time Monitoring

The disclosure researches the calculation formula for the concentration Sᵥ of the carboxylic acids. Through the monitoring and electrical control assembly, the temperature, the pH value, and the water quality inside the fermentation container can be continuously monitored to obtain the monitoring results. The process parameters of these assemblies can be regulated in real time based on the monitoring results to ensure that the fermentation device is in an optimal operation state. The high degree of automation and real-time monitoring function have improved an operation stability and ease of operation of the fermentation device, thereby reducing manual intervention and operational errors.

### 2. Substrate Metabolism Regulation and Greenhouse Gas Emission Reduction

The disclosure combines hydraulic circulation and reagent regulation, thereby achieving uniform disturbance and reflux of the sludge through the hydraulic circulation assembly and improving completeness and efficiency of a fermentation process for the sludge. The substrate metabolism regulation includes regulation of the pH value, the temperature, and the concentrations of the trace elements in the fermentation device, thereby ensuring stability and optimization for a fermentation environment and promoting conversion of the sludge into efficient organic carbon sources (such as carboxylic acids, including acetic acid, propionic acid, butyric acid, and valeric acid). By adding the aforementioned efficient organic carbon sources to the biological reaction tank, abundance of key phosphorus removal microorganisms (such as Flavobacterium, Candidatus Accumul ibacter, and Thaurea, with an increase of 15%-49%) and abundance of key denitrification microorganisms (such as Hyphomicrobium, Terrimonas, and Pseudomonas, with an increase of 6%-12%) in the biological reaction tank can be significantly increased, expression of key genes for denitrification and phosphorus removal (such as encoding polyphosphate kinase (ppk), encoding polyhydroxyalkanoate synthase (phaC), encoding nitric oxide reductase (norC)) are enhanced, the denitrification and phosphorus removal are promoted, regulation of metabolic balance of functional microorganisms in the biological reaction tank is achieved, metabolic activity of non-functional microorganisms (such as the methanogenic bacteria) is inhibited, and greenhouse gas emissions such as methane are avoided, thereby achieving the goal of carbon reeducation.

### 3. Integrated Fermentation and Product Decanting Separation

An integrated design of the fermentation container, the decanting assembly, and the scum skimmer achieves efficient separation and direct utilization of fermentation products. The clarified liquid of the carboxylic acids produced by the fermentation of the sludge is transported to the biological reaction tank through the decanting assembly to promote a denitrification and phosphorus removal process. The excess sludge after the fermentation continues to enter an original sludge treatment process. This design simplifies an operation process, reduces secondary treatment steps, and enables the fermentation products to be directly and efficiently applied to a sewage treatment system.

### 4. Energy-Saving and Environmentally Friendly Insulation Design

The disclosure uses insulation equipment (i.e., the heating and insulation assembly) to maintain the temperature inside the fermentation container at 35° C. to 55° C., fully utilizing heat generated by the fermentation of the sludge without requiring a large amount of additional heat input. The disclosure provides multiple low-energy insulation and heat exchange methods that can be selected according to actual needs. Compared to methods in the art that require high energy consumption and additional heating, the disclosure significantly reduces energy consumption, saves resources, and improves environmental and economic performance of the fermentation device.

### 5. No Need to Add Additional Microbial Strains

The fermentation device with an integrated structure provided by the disclosure can operate automatically after startup without the need for the additional microbial strains and relies on its own system circulation and regulation to achieve efficient fermentation of the sludge. This not only reduces operation costs but also minimizes use of chemical reagents and potential impact of the chemical reagents on environment, making an entire treatment process more environmentally friendly.

### BRIEF DESCRIPTION OF DRAWINGS

In order to more clearly describe embodiments of the disclosure or technical solutions in the art, a brief introduction will be given to attached drawings required for description of the embodiments or the art. Apparently, the attached drawings described below are only part of the embodiments of the disclosure. For those skilled in the art, other attached drawings can be obtained based on these attached drawings without creative labor.

FIG. 1 illustrates a sectional view of a fermentation device for recovering carboxylic acids while reducing greenhouse gas emissions according to an embodiment of the disclosure.

FIG. 2 illustrates a planar view of the fermentation device for recovering the carboxylic acids while reducing the greenhouse gas emissions according to the embodiment of the disclosure.

FIG. 3 illustrates a plan layout diagram of a pipeline of a hydraulic circulation assembly according to the embodiment of the disclosure.

FIG. 4 illustrates a plan layout diagram of pipelines of a sludge inlet assembly and a venting assembly according to the embodiment of the disclosure.

FIG. 5 illustrates a construction diagram of an agitator assembly according to the embodiment of the disclosure.

FIG. 6 illustrates a schematic diagram of the agitator assembly in different work states according to the embodiment of the disclosure.

FIG. 7 illustrates a construction diagram of a decanting assembly according to the embodiment of the disclosure.

FIG. 8 illustrates an elevation angle diagram of blade plates according to embodiment 2 of the disclosure.

FIG. 9 illustrates a process flow of an actual engineering application of the fermentation device for recovering the carboxylic acids while reducing the greenhouse gas emissions according to the embodiment of the disclosure.

FIG. 10 illustrates a diagram of impact of carboxylic acids after being added to a biological reaction tank on key genes for denitrification and phosphorus removal according to the embodiment of the disclosure.

FIG. 11 illustrates a diagram of impact of the carboxylic acids after being added to the biological reaction tank on key phosphorus removal microorganisms according to the embodiment of the disclosure.

FIG. 12 illustrates a diagram of impact of the carboxylic acids after being added to the biological reaction tank on key denitrification microorganisms according to the embodiment of the disclosure.

### DESCRIPTION OF REFERENCE NUMERALS

1: fermentation container; 101: sludge inlet; 102: carboxylic acid outlet; 103: venting port; 104: scum discharge outlet; 105: hydraulic circulation inlet; 106: hydraulic circulation outlet; 107: maintenance manhole; 108: agitator installation hole; 109: exhaust port; 110: breathing port; 111: sampling port; 112: instrument monitoring port; 113: support angle plate; 114: sludge inlet pump; 115: controller;
2: sludge inlet assembly; 21: sludge inlet pipe; 22: sludge inlet pipe bracket; 23: sludge inlet pipe outlet;
3: decanting assembly; 31: decanter; 311: decanting tank; 32: carboxylic acid pipeline; 33: supporting partition plate; 34: scum skimmer; 341: scum skimmer motor; 35: scum collection tank;
4: hydraulic circulation assembly; 41: circulation pump; 42: circulation sludge inlet main pipe; 43: circulation sludge inlet distribution head; 44: circulation sludge inlet branch pipe; 45: circulation sludge inlet pipeline support; 46: circulation sludge outlet pipe; 47: circulation sludge outlet pipe; 48: circulation sludge outlet pipeline support;
5: heating and insulation assembly; 51: heating components; 52: insulation color steel corrugated sheet; 53: insulation ring;
6: agitator assembly; 61: agitator; 611: connection component; 62: multi-stage blade; 621: blade plate; 622: hydraulic support rod; 623: flexible connector; 63: rotating shaft; 631: heat inlet pipe; 632: heat return pipe; 634: electric trace heating cable; 64: shaft end; 65: heat transfer circulation pipeline;
7: reagent regulation assembly; 71: annular reagent distributor; 72: reagent delivery pipe; 73: reagent nozzle; 74: metering pump; 75: reagent box; 76: inert gas storage chamber; 77: inert gas delivery pipe; 78: inert gas blowing nozzle;
8: venting assembly; 81: venting branch pipe; 82: venting intermediate box; 83: venting pipe;
9: monitoring and electrical control assembly; 91: thermometer; 92: pH meter; 93: water quality monitoring probe.

### DETAILED DESCRIPTION OF EMBODIMENTS

Technical solutions of the disclosure will be clearly and completely described as follows with reference to attached drawings. Apparently, embodiments described herein are only a part of embodiments of the disclosure, not all of them. Based on the embodiments of the disclosure, all other embodiments obtained by those skilled in the art without creative labor shall fall within a scope of protection of the disclosure.

In description of the disclosure, it should be noted that directional or positional relationships indicated by terms “center”, “up”, “down”, “left”, “right”, “vertical”, “horizontal”, “inside”, and “outside” are based on directional or positional relationships illustrated in the attached drawings, are only for convenience of describing the technical solutions of the disclosure and simplifying the description, and do not indicate or imply that a device or an element referred to must have a specific orientation, be constructed and operated in a specific orientation, and therefore cannot be understood as limiting the disclosure.

### Embodiment 1

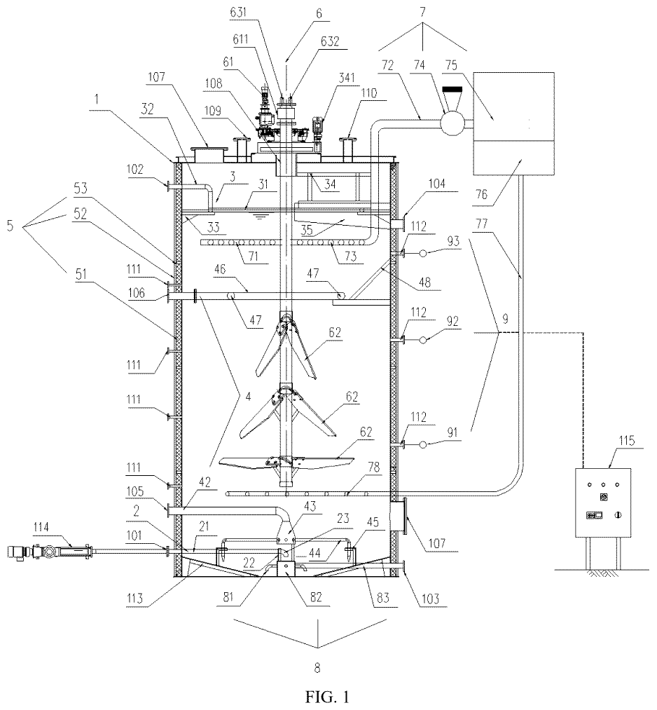

As illustrated in FIG. 1, a fermentation device for recovering carboxylic acids while reducing greenhouse gas emissions provided by the disclosure includes a fermentation container 1, a sludge inlet assembly 2, a decanting assembly 3, a hydraulic circulation assembly 4, a heating and insulation assembly 5, an agitator assembly 6, a reagent regulation assembly 7, a venting assembly 8, and a monitoring and electrical control assembly.

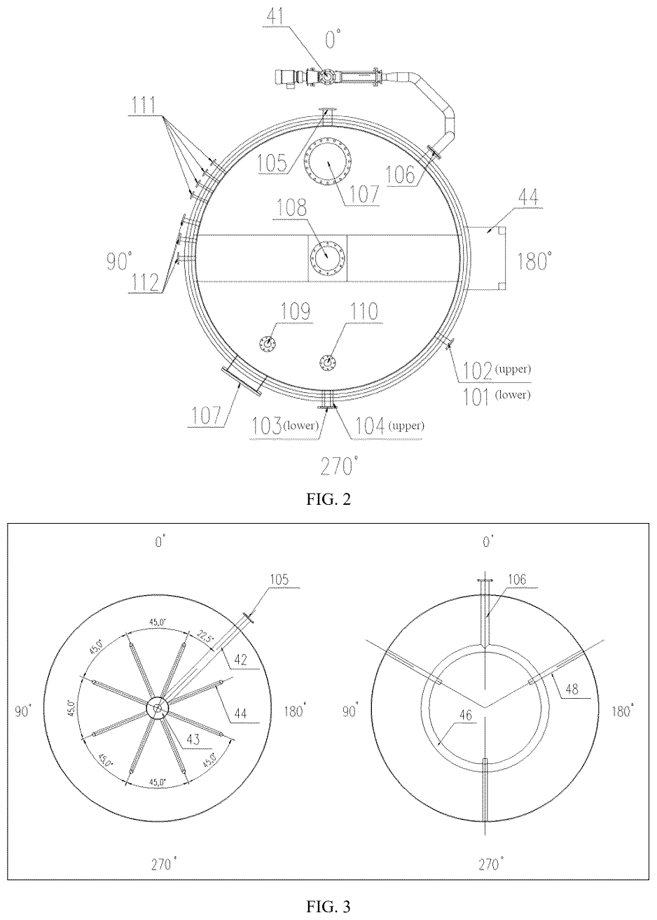

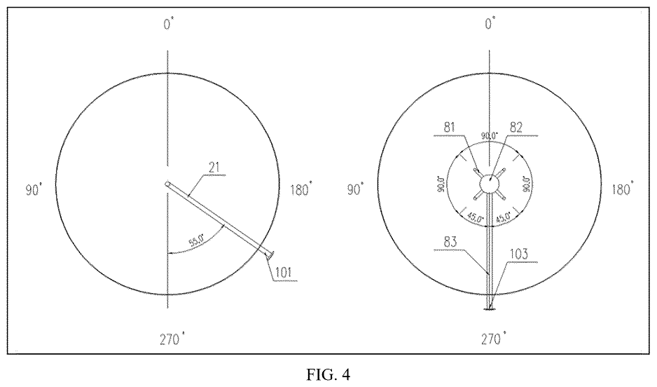

As illustrated in FIG. 2 through FIG. 4, the fermentation container 1 is disposed on ground near a sewage treatment facility, and the fermentation container 1 is used to hold sludge generated by the sewage treatment facility and ferment the sludge into the carboxylic acids that are easily utilized by microorganisms in a biological reaction tank. The sludge inlet assembly 2 is connected to a lower part of the fermentation container 1, and the sludge inlet assembly 2 is used to transport primary sludge or excess sludge generated by the sewage treatment facility into the fermentation container 1. The decanting assembly 3 is disposed inside a top of the fermentation container 1, and the decanting assembly 3 is used to decant the carboxylic acids fermented from the top of the fermentation container 1 and transport the carboxylic acids fermented to the biological reaction tank, thereby promoting a denitrification and phosphorus removal process of the microorganisms in the biological reaction tank. The hydraulic circulation assembly 4 is disposed inside the fermentation container 1, and the hydraulic circulation assembly 4 is used to agitate the sludge inside the fermentation container by suction and recirculation of the sludge through a circulation pump 41, thereby improving a hydraulic circulation flow state of the sludge. The heating and insulation assembly 5 is used to heat the sludge inside fermentation container 1 to a suitable temperature and reduce heat dissipation of the sludge. The agitator assembly 6 is used to fully agitate the sludge in the fermentation container 1 through multi-stage blades 62, ensuring even quality distribution of the sludge. The reagent regulation assembly 7 is used to regulate growth and metabolic environment of the microorganisms in the sludge by delivering reagents into the fermentation container 1, thereby promoting production efficiency of the carboxylic acids during fermentation and improving quality of the carboxylic acids produced. The venting assembly 8 is disposed on a bottom of the fermentation container 1 and is used to discharge all the sludge inside the fermentation container 1 during maintenance for the fermentation device. The monitoring and electrical control assembly 9 is used to continuously monitor the temperature, a pH value, and water quality of the sludge in fermentation container 1 to thereby obtain monitoring results and to regulate process parameters of the sludge inlet assembly 2, the hydraulic circulation assembly 4, the heating and insulation assembly 5, the agitator assembly 6, and the reagent regulation assembly 7 in real time based on the monitoring results, thereby ensuring that the fermentation device is in a best operation condition.

The fermentation container 1 is made of 304 or SS316 stainless steel and has a cylindrical or egg-shaped overall structure. A sludge inlet 101, a hydraulic circulation inlet 105, a venting port 103, and a maintenance manhole 107 are defined on a lower part of a side wall of the fermentation container 1. A hydraulic circulation outlet 106 is defined on a middle-upper part of the side wall of the fermentation container 1. A carboxylic acid outlet 102 and a scum discharge port 104 are defined on an upper part of the side wall of the fermentation container 1. Multiple sampling ports 111 and multiple instrument monitoring ports 112 are uniformly defined on the side wall of the fermentation container 1 from low to high. The multiple sampling ports 111 and the multiple instrument monitoring ports 112 are uniformly distributed along the side wall of the fermentation container 1 at a certain rotation angle from high to low. Each of the multiple sampling ports 111 is connected to a sampling tube, and a shut-off valve is disposed on the sampling tube. A top surface of the fermentation container 1 also defines a maintenance manhole 107, as well as an agitator installation hole 108, an exhaust port 109, and a breathing port 110. The exhaust port 109 is used to discharge gas generated inside the fermentation container 1. The breathing port 110 is used to maintain a pressure balance inside the fermentation container 1. An automatic exhaust check valve is disposed at a top of the exhaust port 109, and the gas generated inside the fermentation container 1 can only be discharged and cannot enter. A reverse intake check valve is disposed at a top of the breathing port 110, allowing gas to only enter the fermentation container 1 and not be discharged. Support angle plates 113 are disposed on the bottom of the fermentation container 1 and are used to firmly support the entire fermentation device on the ground. A tank top plate, tank top reinforced steel bars, and a tank top fence structure are disposed on the top surface of the fermentation container 1, thereby facilitating personnel to inspect the fermentation device through the maintenance manhole 107 on the top surface of the fermentation container 1.

The sludge inlet assembly 2 is disposed on a lower part of the fermentation container 1. The sludge inlet assembly 2 includes a sludge inlet pipe 21, a sludge inlet pump 114 with an adjustable flow rate, and a sludge inlet pipe bracket 22. The sludge inlet pipe 21 is connected to the sludge inlet 101 defined on the lower part of the side wall of the fermentation container 1 and extends into the fermentation container 1 through the sludge inlet 101 until a sludge inlet pipe outlet 23 is located in a central area of the fermentation container 1. The sludge inlet pipe bracket 22 is supported below the sludge inlet pipe 21. The sludge inlet pump 114 is disposed outside the fermentation container 1 and is connected to an inlet of the sludge inlet pipe 21. The sludge pump is configured to pump the primary sludge or the excess sludge generated by the sewage treatment facility. A moisture content of the primary sludge or the excess sludge is 95%-99.5%. A flow rate of the sludge inlet pump 114 can be adjusted according to operation requirements of the fermentation device. The monitoring and electrical control assembly 9 is configured to regulate the flow rate of the sludge inlet pump 114 of the sludge inlet assembly 2 in real time based on the monitoring results and control a fermentation residence duration of the sludge inside the fermentation container 1 in a range of 5 d to 8 d, thereby making a sludge inlet flow rate match a production efficiency of the carboxylic acids.

The decanting assembly 3 is disposed inside the top of the fermentation container 1. The decanting assembly 3 includes a decanter 31, a carboxylic acid pipeline 32, support partition plates 33, a scum skimmer 34, and a scum collection tank 35. The decanter 31 includes a decanting tank 311 in a rectangular or annular shape (see FIG. 7), and a tank opening of the decanting tank 311 is defined inside the top of the fermentation container 1. The carboxylic acids generated by fermentation of the sludge clarify and stratify at a top of the sludge, and clarified liquid of the carboxylic acids at the top of the sludge flows into the decanting tank 311 through the tank opening of the decanting tank 311 in an overflow manner. An end of the carboxylic acid pipeline 32 is connected to a tank body of the decanting tank 311, and another end of the carboxylic acid pipeline 32 is connected to the carboxylic acid outlet 102. The carboxylic acid outlet 102 is connected to the biological reaction tank of the sewage treatment facility to transport the clarified liquid of the carboxylic acids fermented to the biological reaction tank. The scum skimmer 34 is disposed on the top of the fermentation container 1, is driven by a scum skimmer motor 341, and is configured to skim scum on a liquid surface of the clarified liquid of the carboxylic acids into the scum collection tank 35. A tank opening of the scum collection tank 35 is 0.5 cm to 5 cm higher than the liquid surface of the clarified liquid of the carboxylic acids. A tank body of the scum collection tank 35 is inclined downward, and a lowest part of the scum collection tank 35 is connected to the scum discharge port 104. The scum collected is discharged from the fermentation container 1 through the scum discharge port 104.

The hydraulic circulation assembly 4 includes the circulation pump 41, a circulation sludge inlet main pipe 42, a circulation sludge inlet distribution head 43, circulation sludge inlet branch pipes 44, circulation sludge inlet pipeline supports 45, a circulation sludge outlet pipe 46, a circulation sludge outlet 47, and a circulation sludge outlet pipeline support 48. The circulation sludge inlet main pipe 42, the circulation sludge inlet distribution head 43, and the circulation sludge inlet branch pipes 44 are disposed on the bottom of the fermentation container 1 and are sequentially connected in that order. The circulation sludge inlet distribution head 43 is disposed in a central area of the bottom of the fermentation container 1, and the circulation sludge inlet branch pipes 44 are uniformly connected around the circulation sludge inlet distribution head 43 in a radial pattern. The circulation sludge inlet pipeline supports 45 are supported below the circulation sludge inlet branch pipes 44. An end of the circulation sludge inlet main pipe 42 is connected to the circulation sludge inlet distribution head 43, and another end of the circulation sludge inlet main pipe 42 extends outward through the hydraulic circulation inlet 105. The circulation pump 41 is disposed outside the fermentation container 1, an inlet of the circulation pump 41 is connected to an outlet of the circulation sludge inlet main pipe 42, and an outlet of the circulation pump 41 is connected to the circulation sludge outlet pipe 46 located at a middle-upper part of the fermentation container 1. The circulation sludge outlet pipe 46 extends into the fermentation container 1 through the hydraulic circulation outlet 106, and multiple circulation sludge outlets 47 are defined on the circulation sludge outlet pipe 46. During operation, the circulation pump 41 draws the sludge from the bottom of the fermentation container 1 through the circulation sludge inlet branch pipes 44 and the circulation sludge inlet main pipe 42, then pressurizes and pumps the sludge to the circulation sludge outlet pipe 46, and sprays the sludge at high speed through the multiple circulation sludge outlets 47, thereby achieving hydraulic circulation of the sludge within the fermentation container 1 and optimizing a hydraulic flow state of the sludge. The circulation sludge outlet pipeline support 48 is disposed inside the middle-upper part of the fermentation container 1 and is used to support the circulation sludge outlet 47. A working flow rate (with a unit of cubic meters per hour, abbreviated as m³/h) of the circulation pump 41 is taken as 10-50 times a value of “an effective volume (cubic meter, abbreviated as m³) of the fermentation container 1÷24 (hours, abbreviated as h)”, thereby ensuring that under a condition of continuous feeding of the primary sludge and decanting discharge of the carboxylic acids, a residence duration of the sludge in the fermentation container 1 is 5 days.

The heating and insulation assembly 5 includes a heating component 51 and an insulation layer. The heating component 51 is an electric heat tracing cable or a hot water coil pipe heated by a water source heat pump wrapped around at least one of the sludge inlet pipe 21 of the sludge inlet assembly 2 and the side wall of the fermentation container 1, or the heating component 51 is a heating structure disposed inside the fermentation container 1. The insulation layer is disposed on the side wall of the fermentation container 1 and is configured to reduce a heat loss of the fermentation container 1. The insulation layer can be an insulation color steel corrugated sheet 52. The insulation color steel corrugated sheet 52 is disposed on the side wall of fermentation container 1 and fixed by insulation rings 53. The insulation rings 53 are multiple in quantity, and the insulation rings 53 are uniformly disposed outside the fermentation container 1 at certain intervals along a height direction to effectively fix the insulation color steel corrugated sheet 52. The heating structure disposed inside the fermentation container 1 can take various forms, such as heating method one being a heat transfer circulation pipeline form and heating method two being an electric heat tracing cable form. The monitoring and electrical control assembly 9 is configured to regulate the heating and insulation assembly 5 in real time to maintain the temperature of the sludge inside the fermentation container 1 stable in a range of 35° C. to 55° C.

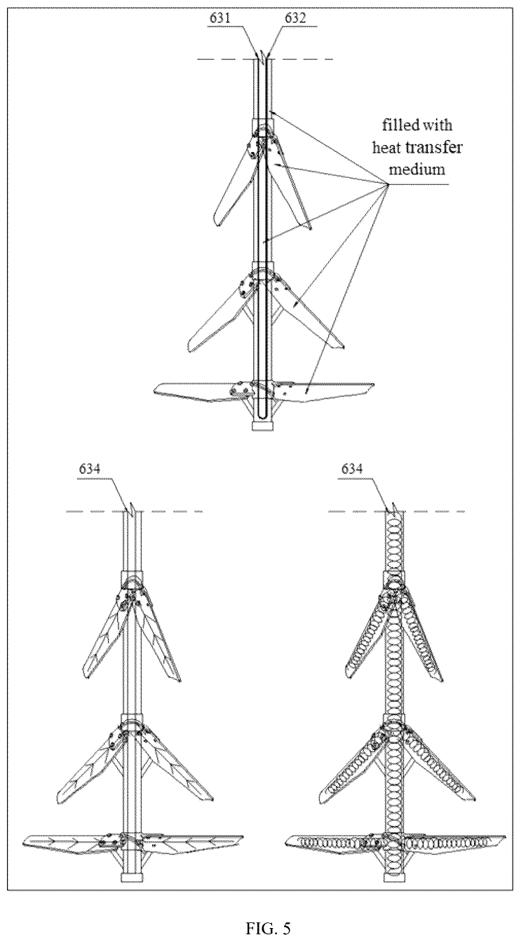

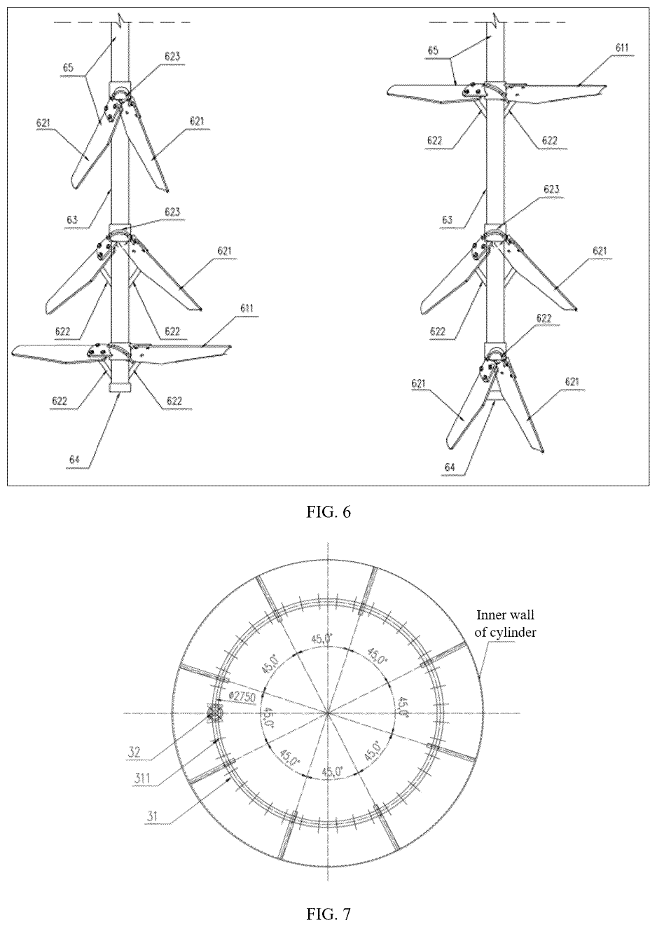

As illustrated in FIG. 1 and FIG. 5 through FIG. 8, the agitator assembly 6 includes an agitator 61 with variable frequency adjustable speed, multi-stage blades 62, a rotating shaft 63, a shaft end 64, and a heat transfer circulation pipeline 65. The agitator 61 is eccentrically disposed in the agitator installation hole 108 defined on the top surface of the fermentation container 1, and the rotating shaft 63 extends into the fermentation container 1. The multi-stage blades 62 are evenly divided into 2-5 layers and disposed inside a middle-lower part of the fermentation container 1. The multi-stage blades 62 are configured to fully agitate the sludge inside the fermentation container 1. The agitator 61 is designed with a variable frequency drive and is connected to the rotating shaft 63 through a connection component 611, allowing rotating speed adjustment as needed. Specifically, a frequency of the agitator 61 can be adjusted variably between 100% and 30%. Preferably, the agitator 61 can dynamically adjust its frequency within a range of 15 hertz (Hz) to 50 Hz. The connection component 611 can be selected as either a rotary joint or a slip ring depending on a heating method. A rotating speed of the multi-stage blades 62 is 30 rpm. The multi-stage blades 62 are uniformly spaced and fixed on the rotating shaft 63, with the shaft end 64 located at a bottom of the rotating shaft 63. Each layer of the multi-stage blades 62 includes multiple blade plates 621, hydraulic support rods 622, and a flexible connector 623. The multiple blade plates 621 are connected to each other by the flexible connector 623, as illustrated in FIG. 6, and angles among the multiple blade plates 621 are adjusted by expansion and contraction of the hydraulic support rods 622. When a larger agitation intensity of the sludge is required, the angles among the multiple blade plates 621 are increased, and when a smaller agitation intensity of the sludge is required, the angles among the multiple blade plates 621 are decreased. In the embodiment, angles among the multiple blade plates 621 of a first layer of the multi-stage blades 62 are 180 degrees (°), angles among the multiple blade plates 621 of a second layer of the multi-stage blades 62 are 90°, and angles among the multiple blade plates 621 of a third layer of the multi-stage blades 62 are 60°. A process of adjusting the angles also has functions of separating impurities and breaking fibrous entanglements.

When the heating structure disposed inside the fermentation container 1 is in the heat transfer circulation pipeline form, the heat transfer circulation pipeline 65 penetrates through the rotating shaft 63 in the agitator assembly 6. The heat transfer circulation pipeline 65 includes a heat inlet pipe 631 and a heat return pipe 632 connected to the heat inlet pipe 631. The rotating shaft 63 and the multi-stage blades 62 are both designed with hollow structures, filled internally with heat transfer oil or water as a heat transfer medium. Hot water or hot steam supplied by the water source heat pump flows into the heat inlet pipe 631. The heat is conducted through the heat transfer medium within the rotating shaft 63 and each of the multiple blade plates 621 of the multi-stage blades 62 to outer surfaces of the rotating shaft 63 and the multiple blade plates 621, and then further conducted to the sludge, achieving internal heating. The heat transfer medium cooled down subsequently flows back to the water source heat pump through the heat return pipe 632. Furthermore, users can select the heat transfer medium based on actual fermentation requirements. Due to properties of heat transfer medium media, heat transfer oil is preferably used as the heat transfer medium for high-temperature fermentation (55° C.), and water is preferably used as the heat transfer medium for medium-temperature fermentation (35° C.). When using this heating method, the agitator 61 is connected to the rotating shaft 63 through the rotary joint, ensuring that rotation of a housing of the rotating shaft 63 does not interfere with the heat transfer circulation pipeline 65.

When the heating structure disposed inside the fermentation container 1 is in the electric heat tracing cable form, electric trace heating cables 634 are wrapped inside the rotating shaft 63 and the multiple blade plates 621, serving as the heat transfer medium. The heat is directly conducted to the outer surfaces of the rotating shaft 63 and the multiple blade plates 621, and then transferred to the sludge, providing internal heating. The electric trace heating cables 634 have multiple arrangement forms: they can be wrapped around middle sections of the rotating shaft 63 and the multiple blade plates 621, or alternatively embedded in a vein-like manner into the middle sections of the rotating shaft 63 and the multiple blade plates 621. Furthermore, the users can select sizes and configurations of the electric trace heating cables 634 according to a volume of the fermentation container 1 and the agitator 61. Specifically, connections between the rotating shaft 63 and the multiple blade plates 621 adopt a flexible connection manner, ensuring that a heating effect is not affected when the multiple blade plates 621 rise and fall. When using this heating method, the agitator 61 is connected to the rotating shaft 63 through the slip ring, so that rotation of rotating shaft 63 does not affect power supply and heat transfer for the electric trace heating cables.

The reagent regulation assembly 7 includes an annular reagent distributor 71, a reagent delivery pipe 72 provided with a metering pump 74, a reagent box 75, an inert gas storage chamber 76, an inert gas delivery pipe 77, and multiple inert gas blowing nozzles 78. The reagent box 75 is disposed outside the fermentation container 1 and is connected to the annular reagent distributor 71 disposed inside the fermentation container 1 through the reagent delivery pipe 72 penetrating through the fermentation container 1. The annular reagent distributor 71 is disposed 20-100 cm below the liquid surface in the fermentation container 1. Multiple reagent nozzles 73 are defined on the annular reagent distributor 71 and are configured to uniformly spray the reagents into the sludge. The reagents in the reagent box 75 include one or more selected from the group consisting of alkaline solution, acidic solution, and trace element solution. The inert gas storage chamber 76 is disposed inside the reagent box 75 and is connected to the inert gas delivery pipe 77. An end portion of the inert gas delivery pipe 77 is horizontally disposed at the bottom of the fermentation container 1. The multiple inert gas blowing nozzles 78 are evenly distributed on the end portion of the inert gas delivery pipe 77. The multiple inert gas blowing nozzles 78 are configured to spray nitrogen, helium, or argon at an appropriate flow rate to blow off residual dissolved oxygen in the sludge, thereby maintaining an anaerobic environment for fermentation of the sludge. The metering pump 74 pumps the reagents in the reagent box 75 to the reagent delivery pipe 72 at an appropriate flow rate. The reagents are evenly sprayed into the sludge through the multiple reagent nozzles 73 distributed on the annular reagent distributor 71, and then thoroughly mixed with the sludge under action of the hydraulic circulation assembly 4 and the agitator assembly 6. The monitoring and electrical control assembly 9 is configured to regulate flow rates of the alkali solution, the acid solution, and the trace element solution in the reagent regulation assembly 7 in real time through the metering pump 74, to thereby maintain the pH value of the sludge inside the fermentation container 1 within a most suitable range of 8-10 and maintain concentrations of trace elements in the sludge as follows: Cu²⁺ in a range of 20 mmol/L to 65 mmol/L, Mn²⁺ in a range of 2.5 mmol/L to 6.0 mmol/L, B³⁺ in a range of 15.0 mmol/L to 28.5 mmol/L, Mo⁶⁺ in a range of 4.0 mmol/L to 6.2 mmol/L, W⁶⁺ in a range of 7.0 mmol/L to 12.2 mmol/L, Ni²⁺ in a range of 3.0 mmol/L to 5.5 mmol/L, Co²⁺ in a range of 10.0 mmol/L to 18.3 mmol/L, Zn²⁺ in a range of 25.5 mmol/L to 35.0 mmol/L, and Ca²⁺ in a range of 10.5 mmol/L to 30.5 mmol/L. Based on years of work experience, the inventor discovers through experiments that the trace elements are key components of active centers of multiple microbial reductases and key genes involved in denitrification and phosphorus removal. These elements have their own functions, and some elements also have synergistic effects. They can improve a hydrolysis process of soluble proteins, fats, and polysaccharides in a fermentation process and promote regulation of microbial intracellular enzyme activity for synthesis of carboxylic acids from hydrolysis products. When the pH value of the sludge in fermentation container 1 is maintained within the most suitable range of 8-10 and the concentrations of the trace elements are maintained in aforementioned ranges, activity of acid producing bacteria (expressed as adenosine triphosphate, abbreviated as ATP) increases by an average of 1.3-1.5 times. Specifically, Cu²⁺, Mn²⁺, B³⁺, Zn²⁺, and Co²⁺ added can effectively promote generation of acetic acid, with a concentration increase of 1.3-1.9 times. Cu²⁺, Mo⁶⁺, Ni²⁺, and Zn²⁺ added can effectively promote generation of propionic acid, with a concentration increase of 1.5-1.8 times. Cu²⁺, Ca²⁺, W⁶⁺, and Co²⁺ added can effectively promote generation of butyric acid, valeric acid, and hexanoic acid, with a concentration increase of 1.1-1.6 times. An overall combination of these trace elements can effectively increase overall concentrations of the carboxylic acids, such as the acetic acid, the propionic acid, the butyric acid, the valeric acid, and the hexanoic acid, and a concentration of the carboxylic acids (measured as COD) is increased by 1.2-1.4 times. This ultimately promotes denitrification and phosphorus removal effects in sewage treatment and reduces accumulation of intermediate products such as greenhouse gases generated during a reaction process. The aforementioned most suitable range of the pH value and concentration ranges of the trace elements have been repeatedly verified in municipal sludge from a sewage treatment plant in Shanghai, a sewage treatment plant in Zhejiang Province, a sewage treatment plant in Jiangsu Province, and a sewage treatment plant in Chongqing, confirming their typicality and universality. The acidic/alkaline solutions and the trace element solution referred to are concentrated liquids, which can be prepared in large quantities once according to demand, avoiding repeated operations. Dosage of these acidic/alkaline solutions and the trace element solution is calculated based on sensor data and logic conversion of an integrated controller.

The venting assembly 8 includes multiple venting branch pipes 81, a venting intermediate box 82, and a venting pipe 83. An inlet of each of the multiple venting branch pipes 81 faces downward, with a distance of 3-30 cm from a bottom surface of the fermentation container 1. The venting intermediate box 82 is a hollow box body, and a side wall of the venting intermediate box 82 is connected to the multiple venting branch pipes 81. An end of the venting pipe 83 is connected to the venting intermediate box 82, and another end of the venting pipe 83 penetrates through fermentation container 1 through the venting port 103. The venting pipe 83 is configured to completely empty the sludge and the carboxylic acids inside the fermentation container during regular sludge discharge and maintenance for the fermentation device.

The monitoring and electrical control assembly 9 includes thermometers 91, pH meters 92, water quality monitoring probes 93, and a controller 115. The thermometers 91, the pH meters 92, and the water quality monitoring probes 93 are respectively embedded into the multiple instrument monitoring ports 112 and are configured to continuously monitor the temperature, the pH value, and the water quality of the sludge inside the fermentation container 1. The water quality monitoring probes 93 can simultaneously monitor concentrations of organic matter (measured as COD), TN, TP, total organic acid anions, acetate, propionate, butyrate, and valerate in the sludge. The controller 115 of the monitoring and electrical control assembly 9 is configured to regulate process parameters of the sludge inlet assembly 2, the hydraulic circulation assembly 4, the heating and insulation assembly 5, the agitator assembly 6, and the reagent regulation assembly 7 in real time based on the monitoring results to ensure that the temperature of the sludge inside the fermentation container 1 is stably maintained in a range of 35° C. to 55° C., thereby producing the carboxylic acids of a predetermined concentration. The fermentation container 1 can adopt a continuous batch mode or a continuous mode, both of which are controlled by the monitoring and electrical control assembly 9 to regulate inflow and outflow of the sludge.

Calculation formulas for the concentrations of the organic matter in the fermentation container 1 and a concentration Sᵥ of the carboxylic acids in reaction hydrolysis products of the sludge are expressed as follows:

$$\begin{matrix}{S}_{v} = \int [ \frac{{N}_{1} + {aN}_{2}}{{N}_{1} + {N}_{2}} \cdot ( \frac{{k}_{m , h} \cdot {X}_{h}}{1 + \frac{{K}_{S , h}}{{S}_{h}} + \frac{{S}_{v}}{{K}_{I , h}}} - \frac{{k}_{m , v} \cdot {X}_{v}}{1 + \frac{{K}_{S , h}}{{S}_{h}} + \frac{{S}_{v}}{{K}_{I , h}}} ) ] dt \\ {S}_{h} = \int [ \frac{{N}_{1} + {aN}_{2}}{{N}_{1} + {N}_{2}} \cdot ( k \cdot {S}_{P} - \frac{{k}_{m , h} \cdot {X}_{h}}{1 + \frac{{K}_{S , h}}{{S}_{h}} + \frac{{S}_{v}}{{K}_{I , h}}} ) ] dt \\ {S}_{P} = \int [ \frac{{N}_{1} + {aN}_{2}}{{N}_{1} + {N}_{2}} \cdot ( - k \cdot {S}_{P} + {k}_{d , h} \cdot {X}_{h} + {k}_{d , v} \cdot {X}_{v} ) ] dt\end{matrix}$$

where Sᵥ represents the concentration of the carboxylic acids produced, measured as COD; Sₚ represents a concentration of the organic matter in sludge to-be-treated, measured as COD; Sₕ represents a concentration of the reaction hydrolysis products, including concentrations of protein, fat, and carbohydrate, measured as COD; N₁ represents a variable frequency output power of the circulation pump 41 for hydraulic stirring; N₂ represents a variable frequency output power of the agitator for mechanical stirring; a represents a regulation coefficient of stirring intensity of the agitator 61; k_{m,h} represents a maximum specific utilization rate of the reaction hydrolysis products; Xₕ represents a microbial concentration of carboxylic acid-producing bacteria; K_{S,h} represents a half-saturation constant for growth of the carboxylic acid-producing bacteria; K_{I,h} represents a influence coefficient of the concentration of the carboxylic acids on the growth of the carboxylic acid-producing bacteria; k_{m,v} represents a maximum specific utilization rate of the carboxylic acids; Xᵥ represents a microbial concentration of methanogenic bacteria; k represents a maximum specific substrate removal rate constant; k_{d,h} represents a microbial endogenous decay rate of the carboxylic acid-producing bacteria; and k_{d,v} represents a microbial endogenous decay rate of the methanogenic bacteria.

The controller 115 of the monitoring and electrical control assembly 9 can calculate the concentration of the carboxylic acids produced using the above formula based on properties of the sludge entering the fermentation container 1 (composition of components such as proteins, carbohydrates, and amino acids), activities and endogenous decay rates of the carboxylic acid-producing bacteria and the methanogenic bacteria, the variable frequency output power, and the regulation coefficient of agitation intensity, etc. At the same time, the controller 115 can also dynamically regulate operation parameters of the fermentation device according to a required concentration of the carboxylic acids produced by the fermentation device. For example, the reaction process inside the fermentation container can be adjusted by adjusting the angles among the multiple blade plates 621 of the agitator assembly 6, or a specific ratio of carboxylic acid mixture (acetic acid concentration: propionic acid concentration of 1:1, 2:1, etc. can be produced as needed to adjust the biological reaction tank) can be produced by adjusting a ratio and concentrations of the proteins, the carbohydrates, and the amino acids in the sludge entering the fermentation container 1. When the acetic acid is a main product of the carboxylic acids recovered (in actual experiments, the acetic acid accounts for up to 78% of total carboxylic acids, while the propionic acid accounts for less than 18%), application of this fermentation device can increase a yield of the acetic acid by an average of 4.5 times compared to technologies in the art and reduce production of greenhouse gases by an average of 82%. When the propionic acid (which accounts for up to 63% of the total carboxylic acid) is the main product of the carboxylic acids recovered, this fermentation device can increase a yield of the propionic acid by an average of 3.2 times compared to the technologies in the art and reduce the production of greenhouse gases by an average of 71%.

The disclosure further provides a fermentation method for recovering the carboxylic acids while reducing the greenhouse gas emissions, including the following steps.

Step 1, the fermentation device for recovering the carboxylic acids while reducing the greenhouse gas emissions provided by the disclosure is installed on the ground near the sewage treatment facility. The fermentation device refers to the above-mentioned fermentation device, which will not be repeated here. There are no special restrictions on a relative orientation and a specific distance between the fermentation device and the sewage treatment facility. The fermentation device can be installed referring to actual terrain for facilitating construction, saving labor, and conserving materials.

Step 2, the sludge inlet assembly 2 connected to the lower part of the fermentation container 1 is used to transport the sludge to-be-treated into the fermentation container 1. The sludge to-be-treated is the primary sludge or the excess sludge generated by the sewage treatment facility, with the moisture content in a range of 95% to 99.5%. The sludge inlet flow rate is regulated according to operation requirements of the fermentation device, and the fermentation residence duration of the sludge in the fermentation container 1 is generally controlled to be 5 d to 8 d.

Step 3, the hydraulic circulation assembly 4 is used to suck and recirculate the sludge in fermentation container 1 to thereby agitate the sludge in fermentation container 1 and improve the hydraulic circulation flow state of the sludge. The heating and insulation assembly 5 is used to heat and insulate the sludge inside fermentation container 1. The agitator assembly 6 is used to fully agitate the sludge inside fermentation container 1, ensuring the even quality distribution of the sludge. The reagent regulation assembly 7 is used to regulate the growth and metabolic environment of the microorganisms in the sludge by delivering the reagents into the fermentation container 1, thereby promoting the production efficiency of the carboxylic acids during fermentation and improving the quality of the carboxylic acids produced.

Specifically, during work, the circulation pump 41 draws the sludge from the bottom of the fermentation container 1 through the circulation sludge inlet branch pipes 44 and the circulation sludge inlet main pipe 42, then pressurizes and pumps the sludge to the circulation sludge outlet pipe 46, and sprays the sludge at high speed through the circulation sludge outlet 47, and the working flow rate (with the unit of m³/h) of the circulation pump 41 is taken as 10-50 times the value of “the effective volume (m³) of the fermentation container 1÷24 (h)”, thereby achieving the hydraulic circulation of the sludge within the fermentation container 1 and optimizing the hydraulic flow state of the sludge.

Step 4, the monitoring and electrical control assembly 9 is used to continuously monitor the temperature, the pH value, and the water quality of the sludge in fermentation container 1 to thereby obtain the monitoring results and to regulate the process parameters of the sludge inlet assembly 2, the hydraulic circulation assembly 4, the heating and insulation assembly 5, the agitator assembly 6, and the reagent regulation assembly 7 in real time based on the monitoring results, thereby ensuring that the fermentation device is in the best working condition to produce the carboxylic acids with a predetermined concentration. The best working condition includes: the fermentation residence duration of the sludge in the fermentation container 1 of 8 d, the temperature of the sludge in the fermentation container maintained at 35° C., the quality distribution of the sludge uniform, the pH value of the sludge in the fermentation container 1 maintained at 10, and the concentrations of the trace elements in the sludge maintained as follows: Cu²⁺ in a range of 20 mmol/L to 65 mmol/L, Mn²⁺ in a range of 2.5 mmol/L to 6.0 mmol/L, B³⁺ in a range of 15.0 mmol/L to 28.5 mmol/L, Mo⁶⁺ in a range of 4.0 mmol/L to 6.2 mmol/L, W⁶⁺ in a range of 7.0 mmol/L to 12.2 mmol/L, Ni²⁺ in a range of 3.0 mmol/L to 5.5 mmol/L, Co²⁺ in a range of 10.0 mmol/L to 18.3 mmol/L, Zn²⁺ in a range of 25.5 mmol/L to 35.0 mmol/L, and Ca²⁺ in a range of 10.5 mmol/L to 30.5 mmol/L.

Step 5, the decanting assembly 3 is used to decant the carboxylic acids fermented from the top of the fermentation container 1 and transport the carboxylic acids fermented to the biological reaction tank, thereby promoting the denitrification and phosphorus removal process.

Specifically, the monitoring and electrical control assembly 9 is used to calculate the concentration of the carboxylic acids produced by the fermentation container 1 using the aforementioned calculation formula based on the process parameters or dynamically adjust the process parameters of the fermentation device according to the predetermined concentration of carboxylic acids.

In the embodiment, an average moisture content of the primary sludge or the excess sludge from the sewage treatment plant actually entering the fermentation device is 97%-98%. The fermentation residence duration of the sludge within the fermentation container 1 is controlled to be 5 d. Meanwhile, the flow rate of the circulation pump 41 in the hydraulic circulation assembly 4 and the rotating speed of the agitator 61 in the agitator assembly 6 are regulated in real time, so that data monitored by the thermometers 91 at different positions tend to be consistent, data monitored by the pH meters 92 at different positions tend to be consistent, and data monitored by the water quality monitoring probes 93 at different positions tend to be consistent. In addition, the heating and insulation assembly 5 is adjusted in real time to maintain the temperature of the sludge inside the fermentation container 1 stably at 35° C. Furthermore, a flow rate of the metering pump 74 in the reagent regulation assembly 7 is regulated in real time to maintain the pH value of the sludge inside the fermentation container 1 at 10 and to maintain the concentrations of trace elements in the sludge as follows: Cu²⁺ 40 mmol/L, Mn²⁺ 3.0 mmol/L, B³⁺ 20.0 mmol/L, Mo⁶⁺ 5.0 mmol/L, W⁶⁺ 9.0 mmol/L, Ni²⁺ 4.0 mmol/L, Co²⁺ 15.0 mmol/L, Zn²⁺ 30.0 mmol/L, and Ca²⁺ 20.0 mmol/L.

The fermentation device for recovering the carboxylic acids while reducing the greenhouse gas emissions according to the embodiment can continuously produce a mixed supernatant of carboxylic acids with a COD concentration of 19,807 milligrams per liter (mg/L). In the mixed supernatant of carboxylic acids, concentrations of the acetate, the propionate, the butyrate, and the valerate are 6,932 mg/L, 3,960 mg/L, 3,896 mg/L, and 3,373 mg/L, respectively. When the carboxylic acids are continuously transported into an anoxic tank inside a reaction tank of the sewage treatment plant with a processing capacity of 15,000 cubic meters per day (m³/d), COD in the anoxic tank is increased by 58.1 mg/L, significantly promoting denitrification and phosphorus removal, saving a large amount of externally added carbon sources. A direct economic benefit generated from saving carbon sources reaches 1.31 million yuan per year, while an indirect economic benefit from reducing emissions of methane and other greenhouse gases reaches 5 million yuan per year.

### Embodiment 2

A fermentation device for recovering carboxylic acids while reducing greenhouse gas emissions provided in the embodiment has the same basic structure and usage method as that provided in embodiment 1, specifically as follows.

The fermentation container 1 is made of 304 or SS316 stainless steel and has a cylindrical or egg-shaped overall structure.

The sludge inlet pump 114 is connected to a front end of the sludge inlet pipe 21. The sludge inlet pump 114 is used to pump municipal sludge or kitchen waste from urban areas. Moisture content of the municipal sludge and the kitchen waste entering the fermentation device is 95% to 97%. Flow rates of the sludge inlet pump 114 and a sludge outlet pump can be adjusted according to operation requirements of the fermentation device, so that under conditions of continuous batch inlet of primary sludge and discharge of the carboxylic acids by decanting water, the residence duration of the sludge in the fermentation container 1 is 8 d, and the temperature of the sludge in the fermentation container 1 is stably maintained at 35° C.

The tank opening of the scum collection tank 35 is 0.5 cm to 5 cm higher than the liquid surface of the clarified liquid of the carboxylic acids.

Forms of the heating component 51 include the electric heat tracing cable or the hot water coil heated by the water source heat pump wrapped around at least one of the sludge inlet pipe 21 of the sludge inlet assembly 2 and the side wall of the fermentation container 1, or the heating component 51 is a heating rod disposed inside the fermentation container 1.

The multi-stage blades 62 are evenly divided into 3 layers and are disposed inside the middle-lower part of the fermentation container 1. The multi-stage blades 62 are configured to fully agitate the sludge inside the fermentation container 1. The heat transfer circulation pipeline 65 conducts the heat into the sludge through the rotating shaft 63 and the multiple blade plates 621 through hot steam circulating therein, thereby heating the sludge.

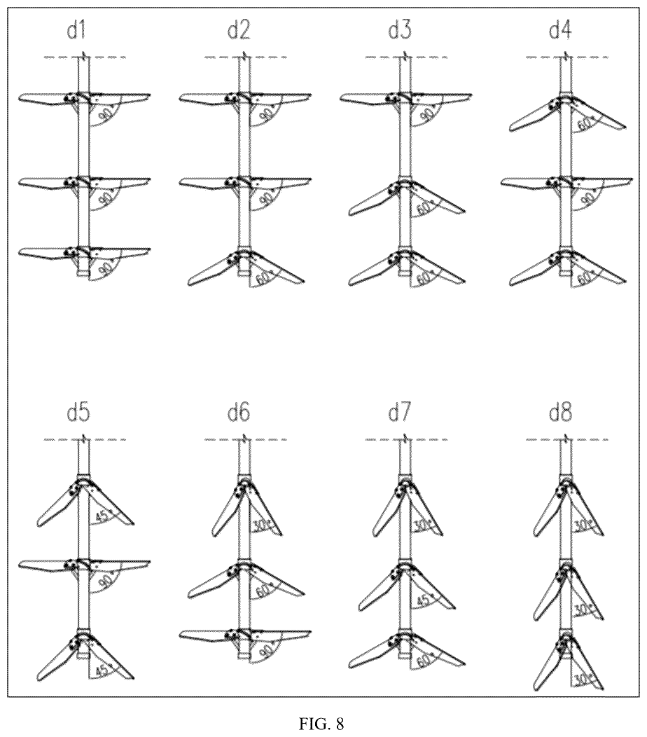

In the embodiment, the multi-stage blades 62 gradually change elevating angles of the multiple blade plates 621 with fermentation time, as illustrated in FIG. 8.

The annular reagent distributor 71 is disposed 20-100 cm below the liquid surface in the fermentation container 1.

The reagents in the reagent box 75 include one or more selected from the group consisting of alkaline solution, acidic solution, and trace element solution. The most suitable pH value in the fermentation container 1 regulated by the alkaline solution and the acidic solution is 10. The concentrations of the trace elements in the fermentation container 1 is controlled by the trace element solution through the metering pump 74 as follows: Cu²⁺ 60 mmol/L, Mn²⁺ 6.0 mmol/L, B³⁺ 28.0 mmol/L, Mo⁶⁺ 6.0 mmol/L, W⁶⁺ 12.0 mmol/L, Ni²⁺ 5.0 mmol/L, Co²⁺ 18.0 mmol/L, Zn²⁺ 35.0 mmol/L, and Ca²⁺ 30.0 mmol/L. The inert gas blowing nozzles 78 are configured to spray the helium at an appropriate flow rate to blow off the residual dissolved oxygen in the sludge, thereby maintaining the anaerobic environment for the fermentation of the sludge.

The inlet of each of the multiple venting branch pipes 81 faces downward, with the distance of 3-30 cm from the bottom surface of the fermentation container 1.

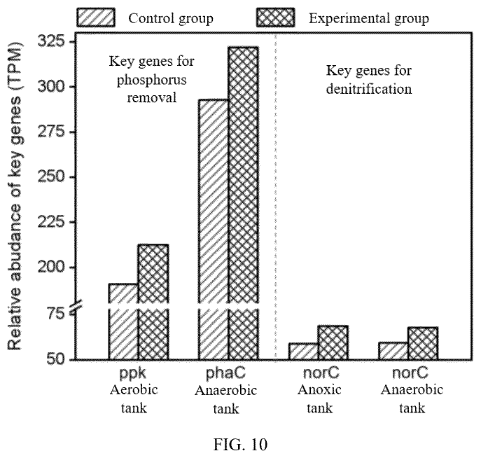

The fermentation device for recovering the carboxylic acids while reducing the greenhouse gas emissions according to the embodiment can continuously produce a mixed supernatant of carboxylic acids with a COD concentration of 37,931 mg/L. In the mixed supernatant of carboxylic acids, the concentrations of the acetate, the propionate, the butyrate, and the valerate are 8,638 mg/L, 8,891 mg/L, 8,327 mg/L, and 8,576 mg/L, respectively. When the carboxylic acids are continuously transported into a reaction tank of a sewage treatment plant with a processing capacity of 50,000 m³/d, expression of key genes for denitrification and phosphorus removal is enhanced. Key genes for phosphorus removal mainly include encoding ppk which catalyzes conversion of ATP into polyphosphate, and encoding phaC which is responsible for synthesis of poly-β-hydroxybutyric acid. Polyphosphate accumulating bacteria provide energy by storing polyhydroxyalkanoate under anaerobic conditions. Key genes for denitrification are encoding norC which reduces nitric oxide (NO) to nitrous oxide (N₂O) under anaerobic or anoxic conditions. As illustrated in FIG. 10, after adding fermentation broth of the sludge, abundance of the two key genes for phosphorus removal significantly increases in both aerobic tank and the anaerobic tank.

After adding the carboxylic acids recovered to the biological reaction tank of the sewage treatment plant, an overall influent COD increases by 59.6 mg/L, significantly promoting the denitrification and phosphorus removal, saving a large amount of externally added carbon sources. A direct economic benefit generated from saving the carbon sources reaches 6.63 million yuan per year, while an indirect economic benefit from reducing emissions of methane and other greenhouse gases reaches 15 million yuan per year.

### Embodiment 3

A fermentation device for recovering carboxylic acids while reducing greenhouse gas emissions provided in the embodiment has the same basic structure and usage method as that provided in embodiment 1, specifically as follows.

The fermentation container 1 is made of 304 stainless steel and has a cylindrical structure.

The sludge inlet pump 114 is connected to a front end of the sludge inlet pipe 21. The sludge inlet pump 114 is used to pump the primary sludge or the excess sludge in the sewage treatment facility. Moisture content of the primary sludge or the excess sludge is 98%. The flow rate of the sludge inlet pump 114 can be adjusted according to operation requirements of the fermentation device, so that under conditions of continuous inlet of the primary sludge and discharge of the carboxylic acids by decanting water, the residence duration of the sludge in the fermentation container 1 is 6 d, and the temperature of the sludge in the fermentation container 1 is stably maintained at 35° C.

The tank opening of the scum collection tank 35 is 3 cm higher than the liquid surface of the clarified liquid of the carboxylic acids.

The heating component 51 is a heating rod disposed inside the fermentation container 1.

The multi-stage blades 62 are evenly divided into 2-5 layers and are disposed inside the middle-lower part of the fermentation container 1.

The annular reagent distributor 71 is disposed 20-100 cm below the liquid surface in the fermentation container 1.

The reagents in the reagent box 75 include one or more selected from the group consisting of alkaline solution, acidic solution, and trace element solution. The most suitable pH value in the fermentation container 1 regulated by the alkaline solution and the acidic solution is 10. The concentrations of the trace elements in the fermentation container 1 are controlled by the trace element solution through the metering pump 74 as follows: Cu²⁺ 65 mmol/L, Mn²⁺ 2.5 mmol/L, B³⁺ 15.0 mmol/L, Co²⁺ 10.0 mmol/L, and Zn²⁺ 25.5 mmol/L. The multiple inert gas blowing nozzles 78 are configured to spray the helium at an appropriate flow rate to blow off the residual dissolved oxygen in the sludge, thereby maintaining the anaerobic environment for the fermentation of the sludge.

The inlet of each of the multiple venting branch pipes 81 faces downward, with the distance of 20 cm from the bottom surface of the fermentation container 1.

The fermentation device for recovering the carboxylic acids while reducing the greenhouse gas emissions according to the embodiment can continuously produce mixed supernatant of carboxylic acids with a COD concentration of 28,971 mg/L. In the mixed supernatant of carboxylic acids, the concentrations of the acetate, the propionate, the butyrate, and the valerate are 10,138 mg/L, 8,691 mg/L, 2,657 mg/L, and 2,877 mg/L, respectively.

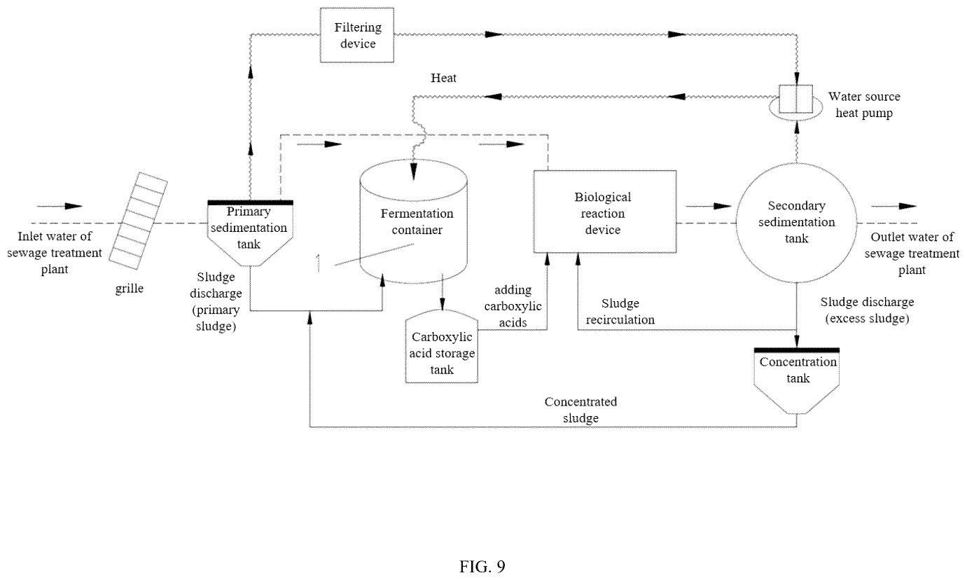

A process flow of an application method of the fermentation device for recovering the carboxylic acids while reducing the greenhouse gas emissions according to the embodiment is illustrated in FIG. 9. The fermentation device collects all primary sludge and excess sludge from a primary sedimentation tank and a sludge concentration tank of the sewage treatment plant for fermentation and recovery of carboxylic acids. The reaction parameters are controlled to avoid production of methane and other greenhouse gases during the fermentation of the sludge. The carboxylic acids produced are added to the biological reaction tank to achieve coordinated removal of pollutants such as COD, nitrogen (N), and phosphorus (P), and the greenhouse gases such as carbon dioxide (CO₂), N₂O, and NO.

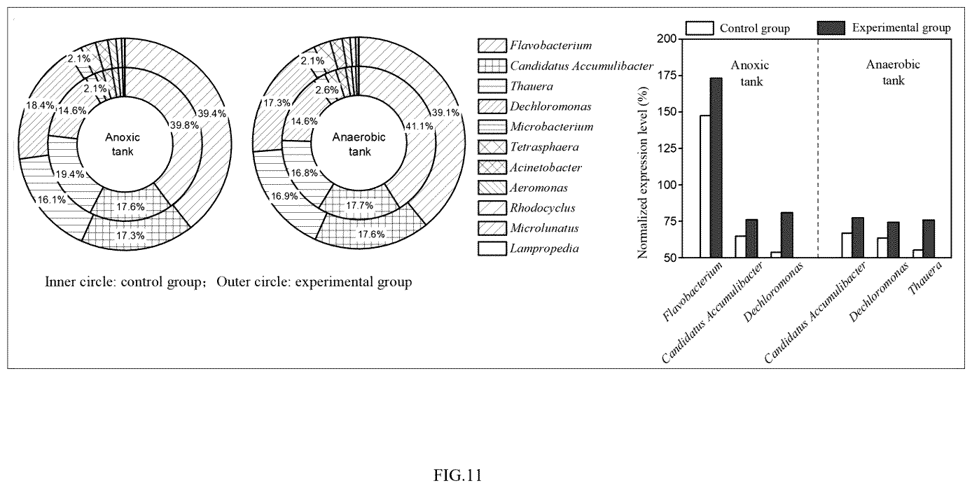

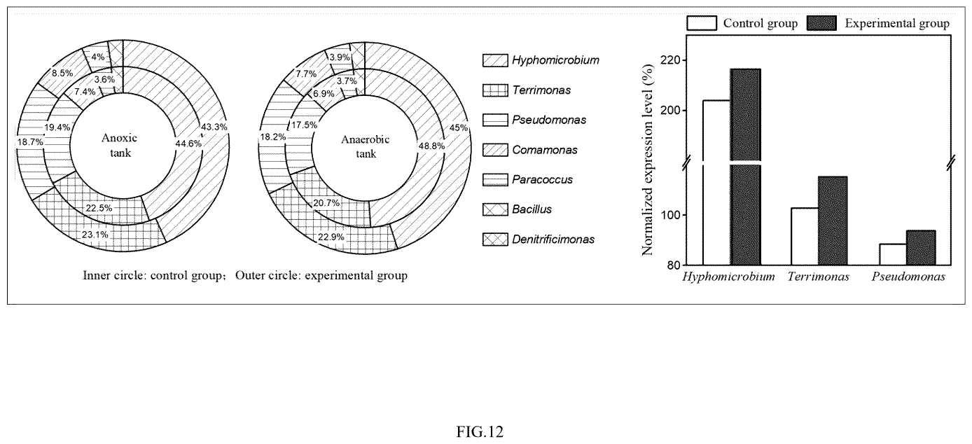

As illustrated in FIG. 11, the carboxylic acids are continuously transported into biological reaction tanks (including the anaerobic tank, an anoxic tank, and the aerobic tank) of a sewage treatment plant with a processing capacity of 30,000 m³/d, after adding fermentation broth of the sludge, abundance of key phosphorus removal microorganisms in the anaerobic tank is increased significantly, and abundances of Flavobacterium, Candidatus Accumulibacter, Thaurea, and Dechloromonas are increased by 115% to 149% compared with a control group. At the same time, adding the carboxylic acids significantly improves abundance of denitrification microorganisms. Main denitrification microorganisms include Hyphomicrobium, Terrimonas, and Pseudomonas. As illustrated in FIG. 12, after adding the fermentation broth of the sludge, abundances of key denitrification microorganisms, including Hyphomicrobium, Terrimonas, and Pseudomonas, in the biological reaction tanks are increased by 106% to 112% compared to the control group. The carboxylic acids generated by a new process achieve clean and efficient treatment of sewage biological reactions. Fermentation broth containing a certain amount of propionic acid (which accounts for more than 30% of the total carboxylic acids) can achieve better denitrification and phosphorus removal rates (an average increase of 25.4% and 31.3%, respectively), with less CO₂, N₂O, NO, and sludge production (an average decrease of 71.1%, 65.3%, 23.4%, and 43.1%, respectively), reducing chemical carbon source input of the sewage treatment plant by 38-100%. A principle of this process is as follows: the fermentation broth contains trace elements such as propionic acid and copper ions. The propionic acid promotes synthesis of more poly hydroxy valerate (PHV) by functional microorganisms during an anaerobic stage, which leads to a better match between rates of energy and reducing power generation from oxidation and decomposition of PHV during aerobic and anoxic stages and rates of phosphorus uptake and denitrification. Consequently, it enhances phosphorus absorption and denitrification while reducing production of CO₂, N₂O, and NO. The copper ions are key components of active centers of various denitrification reductases, which promote denitrification and reduce accumulation of intermediate products. Adding the carboxylic acids can improve COD of the biological reaction tank by 57.2 mg/L, significantly promoting the denitrification and phosphorus removal, saving a large amount of externally added carbon sources. A direct economic benefit generated from saving the carbon sources reaches 3.23 million yuan per year, while an indirect economic benefit from reducing emissions of methane and other greenhouse gases reaches 8 million yuan per year.

TABLE 1
Experimental parameters for each embodiment (unit of concentrations of the trace elements: mmol/L)

| | fermentation | | | | | | | | | | | |
|---|---|---|---|---|---|---|---|---|---|---|---|---|
| | residence | | | | | | | | | | pH | |
| Group | duration | Cu²⁺ | Mn²⁺ | B³⁺ | Mo⁶⁺ | W⁶⁺ | Ni²⁺ | Co²⁺ | Zn²⁺ | Ca²⁺ | value | Temperature |
| Embodiment | 5 d | 40 | 3.0 | 20.0 | 5.0 | 9.0 | 4.0 | 15.0 | 30.0 | 20.0 | 10 | 35° C. |
| 1 | | | | | | | | | | | | |
| Control | 5 d | — | — | — | — | — | — | — | — | — | — | 35° C. |
| group 1 | | | | | | | | | | | | |
| Embodiment | 8 d | 60 | 6.0 | 28.0 | 6.0 | 12.0 | 5.0 | 18.0 | 35.0 | 30.0 | 10 | 35° C. |
| 2 | | | | | | | | | | | | |
| Control | 8 d | — | — | — | — | — | — | — | — | — | — | 35° C. |
| group 2 | | | | | | | | | | | | |
| Embodiment | 6 d | 65 | 2.5 | 15.0 | 4.0 | 7.0 | 3.0 | 10.0 | 25.5 | 10.5 | 10 | 35° C. |
| 3 | | | | | | | | | | | | |
| Control | 6 d | — | — | — | — | — | — | — | — | — | — | 35° C. |
| group 3 | | | | | | | | | | | | |

TABLE 2
Experimental results for each embodiment and each
control group (unit of concentration: mg/L)

| Experimental result | COD | acetate | propionate | butyrate | valerate |
|---|---|---|---|---|---|
| Embodiment 1 | 19807 | 6932 | 3960 | 3896 | 3373 |
| Control group 1 | 14231 | 3952 | 2643 | 3247 | 2195 |
| Embodiment 2 | 37931 | 8638 | 8891 | 8327 | 8576 |
| Control group 2 | 26094 | 5853 | 6231 | 6127 | 6098 |
| Embodiment 3 | 28971 | 10138 | 8691 | 2657 | 2877 |
| Control group 3 | 20155 | 6453 | 5337 | 2319 | 2501 |

To sum up, the fermentation device for recovering the carboxylic acids while reducing the greenhouse gas emissions provided by the disclosure is used to collect and treat all primary sludge and excess sludge from the sewage treatment plant for fermentation and recovery of the carboxylic acids. The fermentation device can also mix urban kitchen waste and the sludge for fermentation to produce the carboxylic acids. By operation of the fermentation device and controlling the reaction parameters, a specific ratio of carboxylic acid mixture is generated, significantly reducing the greenhouse gas emissions, such as methane produced by fermentation. The carboxylic acid mixture recovered can be used to add to the biological reaction tank of the sewage treatment plant for clean ultra-purification treatment of sewage, as well as synthetic efficient removal of greenhouse gases such as CO₂, N₂O, NO during the reaction process. Carboxylic acid industrial products can also be purified and recovered by separating supernatant to achieve resource recovery.

Compared with effects of commonly purchased acetic acid chemicals used as supplementary carbon sources in sewage treatment plants, the fermentation broth containing a certain amount of propionic acid (which accounts for more than 30% of the total carboxylic acids) produced by the fermentation device of the disclosure using organic wastes such as the sludge and kitchen waste can achieve better denitrification and phosphorus removal rates (an average increase of 25.4% and 31.3%, respectively), with less CO₂, N₂O, and sludge production (an average decrease of 71.1%, 23.4%, and 43.1%, respectively). A mechanism by which an increased proportion of propionic acid significantly improves sewage treatment efficiency is as follows: the propionic acid promotes synthesis of more PHV by functional microorganisms for sewage treatment during the anaerobic stage, which leads to a better match between the rates of energy and reducing power generation from oxidation and decomposition of PHV during aerobic and anoxic stages and the rates of phosphorus uptake and denitrification. Consequently, it enhances phosphorus absorption and denitrification while reducing production of CO₂ and N₂O.

Compared with the related art, the fermentation device for recovering the carboxylic acids while reducing the greenhouse gas emissions provided by the disclosure has at least the following beneficial effects.

### 1. Substrate Metabolism Regulation and Greenhouse Gas Emission Reduction

The disclosure combines hydraulic circulation and reagent regulation, thereby achieving uniform disturbance and reflux of the sludge through the hydraulic circulation assembly and improving completeness and efficiency of the fermentation process for the sludge. The substrate metabolism regulation includes regulation of the pH value, the temperature, and the concentrations of the trace elements in the fermentation device, thereby ensuring stability and optimization for a fermentation environment and promoting conversion of the sludge into efficient organic carbon sources (such as carboxylic acids, including acetic acid, propionic acid, butyric acid, and valeric acid). By adding the aforementioned efficient organic carbon sources to the biological reaction tank, abundance of key phosphorus removal microorganisms (such as Flavobacterium, Candidatus Accumul ibacter, and Thaurea, with an increase of 15%-49%) and abundance of key denitrification microorganisms (such as Hyphomicrobium, Terrimonas, and Pseudomonas, with an increase of 6%-12%) in the biological reaction tank can be significantly increased, expression of key genes for denitrification and phosphorus removal (such as encoding ppk, encoding phaC, encoding norC) are enhanced, the denitrification and phosphorus removal are promoted, regulation of metabolic balance of functional microorganisms in the biological reaction tank is achieved, metabolic activity of non-functional microorganisms (such as methanogenic bacteria) is inhibited, and greenhouse gas emissions such as methane are avoided, thereby achieving the goal of carbon reeducation.

### 2. Integrated Fermentation and Product Decanting Separation

An integrated design of the fermentation container, the decanting assembly, and the scum skimmer achieves efficient separation and direct utilization of fermentation products. The clarified liquid of the carboxylic acids produced by the fermentation of the sludge is transported to the biological reaction tank through the decanting assembly to promote the denitrification and phosphorus removal process. The excess sludge after the fermentation continues to enter an original sludge treatment process. This design simplifies an operation process, reduces secondary treatment steps, and enables the fermentation products to be directly and efficiently applied to a sewage treatment system.

### 3. Energy-Saving and Environmentally Friendly Insulation Design

The disclosure uses insulation equipment (i.e., the heating and insulation assembly) to maintain the temperature inside the fermentation container at 35° C. to 55° C., fully utilizing heat generated by the fermentation of the sludge without requiring a large amount of additional heat input. The disclosure provides multiple low-energy insulation and heat exchange methods that can be selected according to actual needs. Compared to methods in the art that require high energy consumption and additional heating, the disclosure significantly reduces energy consumption, saves resources, and improves environmental and economic performance of the fermentation device.

### 4. High Degree of Automation and Real-Time Monitoring

The disclosure researches the calculation formula for the concentration Sᵥ of the carboxylic acids. Through the monitoring and electrical control assembly, the temperature, the pH value, and the water quality inside the fermentation container can be continuously monitored to obtain the monitoring results. The process parameters of these assemblies can be regulated in real time based on the monitoring results to ensure that the fermentation device is in an optimal operation state. The high degree of automation and real-time monitoring function have improved a work stability and ease of operation of the fermentation device, reducing manual intervention and operational errors.

### 5. No Need to Add Additional Microbial Strains

The fermentation device with an integrated structure provided by the disclosure can operate automatically after startup without the need for the additional microbial strains and relies on its own system circulation and regulation to achieve efficient fermentation of the sludge. This not only reduces operation costs but also minimizes use of chemical reagents and potential impact of the chemical reagents on environment, making an entire treatment process more environmentally friendly.

Those skilled in the art can make various other corresponding changes and deformations according to the technical solutions and concepts described above, and all these changes and deformations shall fall within the scope of protection of the claims of the disclosure.

What is claimed is:

1. A fermentation device for recovering carboxylic acids while reducing greenhouse gas emissions, comprising a fermentation container (1), a sludge inlet assembly (2), a decanting assembly (3), a hydraulic circulation assembly (4), a heating and insulation assembly (5), an agitator assembly (6), a reagent regulation assembly (7), and a monitoring and electrical control assembly (9); and
wherein a sludge inlet (101) and a hydraulic circulation inlet (105) are defined on a lower part of a side wall of the fermentation container (1), a hydraulic circulation outlet (106) is defined on a middle-upper part of the side wall of the fermentation container (1), and a carboxylic acid outlet (102) is defined on an upper part of the side wall of the fermentation container (1); the sludge inlet assembly (2) is connected to the sludge inlet (101) and extends into an interior of the fermentation container (1); the decanting assembly (3) is disposed inside a top of the fermentation container (1), and the decanting assembly (3) is connected to the carboxylic acid outlet (102) to decant the carboxylic acids fermented from the top of the fermentation container (1) and transport the carboxylic acids fermented to a biological reaction tank; the hydraulic circulation assembly (4) is connected to the hydraulic circulation inlet (105) and the hydraulic circulation outlet (106), and the hydraulic circulation assembly (4) is configured to agitate sludge inside the fermentation container (1) by suction and recirculation for the sludge through a circulation pump (41); the heating and insulation assembly (5) is disposed on the side wall of the fermentation container (1) and is configured to heat and insulate the sludge inside the fermentation container (1); the agitator assembly (6) is configured to fully agitate the sludge through multi-stage blades (62) disposed inside the fermentation container (1); the reagent regulation assembly (7) is connected to the fermentation container (1) and is configured to deliver reagents into the fermentation container (1); the monitoring and electrical control assembly (9) comprises a controller (115) and monitoring instruments disposed on the fermentation container (1), the monitoring instruments are configured to continuously monitor a temperature, a potential of hydrogen (pH) value, and water quality of the sludge inside the fermentation container (1), to obtain monitoring results, and the controller (115) is configured to regulate process parameters of the sludge inlet assembly (2), the hydraulic circulation assembly (4), the heating and insulation assembly (5), the agitator assembly (6), and the reagent regulation assembly (7) in real time based on the monitoring results to ensure that the temperature of the sludge inside the fermentation container (1) is stably maintained in a range of 35 degree Celsius (C) to 55° C., thereby producing the carboxylic acids of a predetermined concentration; and a calculation formula for a concentration Sᵥ of the carboxylic acids is expressed as follows:
$$\begin{matrix}{S}_{v} = \int [ \frac{{N}_{1} + {aN}_{2}}{{N}_{1} + {N}_{2}} \cdot ( \frac{{k}_{m , h} \cdot {X}_{h}}{1 + \frac{{K}_{S , h}}{{S}_{h}} + \frac{{S}_{v}}{{K}_{I , h}}} - \frac{{k}_{m , v} \cdot {X}_{v}}{1 + \frac{{K}_{S , h}}{{S}_{h}} + \frac{{S}_{v}}{{K}_{I , h}}} ) ] dt \\ {S}_{h} = \int [ \frac{{N}_{1} + {aN}_{2}}{{N}_{1} + {N}_{2}} \cdot ( k \cdot {S}_{P} - \frac{{k}_{m , h} \cdot {X}_{h}}{1 + \frac{{K}_{S , h}}{{S}_{h}} + \frac{{S}_{v}}{{K}_{I , h}}} ) ] dt \\ {S}_{P} = \int [ \frac{{N}_{1} + {aN}_{2}}{{N}_{1} + {N}_{2}} \cdot ( - k \cdot {S}_{P} + {k}_{d , h} \cdot {X}_{h} + {k}_{d , v} \cdot {X}_{v} ) ] dt\end{matrix}$$
where Sᵥ represents the concentration of the carboxylic acids, measured as chemical oxygen demand (COD); Sₚ represents a concentration of organic matter in sludge to-be-treated, measured as COD; Sₕ represents a concentration of reaction hydrolysis products, comprising concentrations of protein, fat, and carbohydrate, measured as COD; N₁ represents a variable frequency output power of the circulation pump (41) for hydraulic agitation; N₂ represents a variable frequency output power of an agitator (61) for mechanical agitation; a represents a regulation coefficient of agitation intensity of the agitator (61); k_{m,h} represents a maximum specific utilization rate of the reaction hydrolysis products; Xₕ represents a microbial concentration of carboxylic acid-producing bacteria; K_{S,h} represents a half-saturation constant for growth of the carboxylic acid-producing bacteria; K_{I,h} represents an influence coefficient of the concentration of the carboxylic acids on the growth of the carboxylic acid-producing bacteria; k_{m,v} represents a maximum specific utilization rate of the carboxylic acids; Xᵥ represents a microbial concentration of methanogenic bacteria; k represents a maximum specific substrate removal rate constant; k_{d,h} represents a microbial endogenous decay rate of the carboxylic acid-producing bacteria; and k_{d,v} represents a microbial endogenous decay rate of the methanogenic bacteria.

2. The fermentation device for recovering the carboxylic acids while reducing the greenhouse gas emissions as claimed in claim 1, wherein the sludge inlet assembly (2) comprises a sludge inlet pipe (21), a sludge inlet pump (114) with an adjustable flow rate, and a sludge inlet pipe bracket (22); the sludge inlet pipe (21) penetrates through the sludge inlet (101) on the side wall of the fermentation container (1) and extends into the fermentation container (1) until a sludge inlet pipe outlet (23) is located in a central area of the fermentation container (1); the sludge inlet pipe bracket (22) is supported below the sludge inlet pipe (21); the sludge inlet pump (114) is connected to an inlet of the sludge inlet pipe (21) and is configured to pump primary sludge or excess sludge generated by an external sewage treatment facility, and the monitoring and electrical control assembly (9) is configured to regulate the flow rate of the sludge inlet pump (114) of the sludge inlet assembly (2) in real time based on the monitoring results and control a fermentation residence duration of the sludge inside the fermentation container (1) in a range of 5 days (d) to 8 d, thereby making a sludge inlet flow rate match a production efficiency of the carboxylic acids.

3. The fermentation device for recovering the carboxylic acids while reducing the greenhouse gas emissions as claimed in claim 1, wherein a scum discharge port (104) is defined on the upper part of the side wall of the fermentation container (1), the carboxylic acid outlet (102) is connected to the biological reaction tank of an external sewage treatment facility to transport clarified liquid of the carboxylic acids fermented to the biological reaction tank; the decanting assembly (3) comprises a decanter (31), a carboxylic acid pipeline (32), a scum skimmer (34), and a scum collection tank (35); the decanter (31) comprises a decanting tank (311) in a rectangular or annular shape, and a tank opening of the decanting tank (311) is defined inside the top of the fermentation container (1); the carboxylic acids generated by fermentation of the sludge clarifies and stratifies at a top of the sludge, and the clarified liquid of the carboxylic acids at the top of the sludge flows into the decanting tank (311) through the tank opening of the decanting tank (311) in an overflow manner; an end of the carboxylic acid pipeline (32) is connected to a tank body of the decanting tank (311), and another end of the carboxylic acid pipeline (32) is connected to the carboxylic acid outlet (102); the scum skimmer (34) is disposed on the top of the fermentation container (1), is driven by a scum skimmer motor (341), and is configured to skim scum on a liquid surface of the clarified liquid of the carboxylic acids into the scum collection tank (35); a tank opening of the scum collection tank (35) is 0.5 centimeters (cm) to 5 cm higher than the liquid surface of the clarified liquid of the carboxylic acids, a tank body of the scum collection tank (35) is inclined downward, and a lowest part of the scum collection tank (35) is connected to the scum discharge port (104); and the scum collected is discharged from the fermentation container (1) through the scum discharge port (104).

4. The fermentation device for recovering the carboxylic acids while reducing the greenhouse gas emissions as claimed in claim 1, wherein the hydraulic circulation assembly (4) comprises the circulation pump (41), a circulation sludge inlet main pipe (42), a circulation sludge inlet distribution head (43), circulation sludge inlet branch pipes (44), and a circulation sludge outlet pipe (46); the circulation sludge inlet distribution head (43) is disposed in a central area of a bottom of the fermentation container (1), and the circulation sludge inlet branch pipes (44) are uniformly connected around the circulation sludge inlet distribution head (43) in a radial pattern; an end of the circulation sludge inlet main pipe (42) is connected to the circulation sludge inlet distribution head (43), and another end of the circulation sludge inlet main pipe (42) extends outward through the hydraulic circulation inlet (105); the circulation pump (41) is disposed outside the fermentation container (1), an inlet of the circulation pump (41) is connected to an outlet of the circulation sludge inlet main pipe (42), and an outlet of the circulation pump (41) is connected to the circulation sludge outlet pipe (46) located at a middle-upper part of the fermentation container (1); the circulation sludge outlet pipe (46) extends into the fermentation container (1) through the hydraulic circulation outlet (106), and a plurality of circulation sludge outlets (47) are defined on the circulation sludge outlet pipe (46).

5. The fermentation device for recovering the carboxylic acids while reducing the greenhouse gas emissions as claimed in claim 1, wherein the heating and insulation assembly (5) comprises a heating component (51) and an insulation layer; the heating component (51) is an electric heat tracing cable or a hot water coil heated by a water source heat pump wrapped around at least one of the sludge inlet pipe (21) of the sludge inlet assembly (2) and the side wall of the fermentation container (1), or the heating component (51) is a heating structure disposed inside the fermentation container (1); the insulation layer is disposed on the side wall of the fermentation container (1) and is configured to reduce a heat loss of the fermentation container (1); and the monitoring and electrical control assembly (9) is configured to regulate the heating and insulation assembly (5) in real time to maintain the temperature of the sludge inside the fermentation container (1) stable in a range of 35° C. to 55° C.

6. The fermentation device for recovering the carboxylic acids while reducing the greenhouse gas emissions as claimed in claim 1, wherein a top surface of the fermentation container (1) defines an agitator installation hole (108); the agitator assembly (6) comprises the agitator (61) with a variable frequency adjustable speed, the multi-stage blades (62), a rotating shaft (63), and a heat transfer circulation pipeline (65); the agitator (61) is eccentrically disposed in the agitator installation hole (108), and the rotating shaft (63) extends into the fermentation container (1); the multi-stage blades (62) are disposed on a middle-lower part of the rotating shaft (63) and are evenly divided into 2-5 layers, and the multi-stage blades (62) are configured to fully agitate the sludge inside the fermentation container (1); a rotating speed of the multi-stage blades (62) is 20 revolutions per minute (rpm) to 60 rpm; each layer of the multi-stage blades (62) comprises a plurality of blade plates (621), hydraulic support rods (622), and a flexible connector (623), the plurality of blade plates (621) are connected to each other by the flexible connector (623), and angles among the plurality of blade plates (621) are adjusted by expansion and contraction of the hydraulic support rods (622); when a larger agitation intensity of the sludge is required, the angles among the plurality of blade plates (621) are increased; and when a smaller agitation intensity of the sludge is required, the angles among the plurality of blade plates (621) are decreased.

7. The fermentation device for recovering the carboxylic acids while reducing the greenhouse gas emissions as claimed in claim 6, wherein the heat transfer circulation pipeline (65) penetrates through the rotating shaft (63) and the plurality of blade plates (621); the heat transfer circulation pipeline (65) comprises a heat inlet pipe (631) and a heat return pipe (632) connected to the heat inlet pipe (631); the heat inlet pipe (631) is configured to transport heat transfer medium supplied by a water source heat pump to thereby conduct heat to the sludge through the rotating shaft (63) and the plurality of blade plates (621); and the heat return pipe (632) is configured to transport cooling water back to the water source heat pump.

8. The fermentation device for recovering the carboxylic acids while reducing the greenhouse gas emissions as claimed in claim 1, wherein the reagent regulation assembly (7) comprises a reagent distributor, a reagent delivery pipe (72) provided with a metering pump (74), and a reagent box (75); the reagent box (75) is disposed outside the fermentation container (1) and is connected to the reagent distributor disposed inside the fermentation container (1) through the reagent delivery pipe (72) penetrating through the fermentation container (1); the reagents in the reagent box (75) comprise one or more selected from the group consisting of alkaline solution, acidic solution, and trace element solution; the monitoring and electrical control assembly (9) is configured to regulate flow rates of the alkali solution, the acid solution, and the trace element solution in the reagent regulation assembly (7) in real time through the metering pump (74), to thereby maintain the pH value of the sludge inside the fermentation container (1) at 8-10 and maintain concentrations of trace elements in the sludge as follows: copper ion (Cu²⁺) in a range of 20 millimoles per liter (mmol/L) to 65 mmol/L, manganese ion (Mn²⁺) in a range of 2.5 mmol/L to 6.0 mmol/L, boron ion (B³⁺) in a range of 15.0 mmol/L to 28.5 mmol/L, molybdenum ion (Mo⁶⁺) in a range of 4.0 mmol/L to 6.2 mmol/L, wolframium ion (W⁶⁺) in a range of 7.0 mmol/L to 12.2 mmol/L, nickel ion (Ni²⁺) in a range of 3.0 mmol/L to 5.5 mmol/L, cobalt ion (Co²⁺) in a range of 10.0 mmol/L to 18.3 mmol/L, zinc ion (Zn²⁺) in a range of 25.5 mmol/L to 35.0 mmol/L, and calcium ion (Ca²⁺) in a range of 10.5 mmol/L to 30.5 mmol/L.

9. The fermentation device for recovering the carboxylic acids while reducing the greenhouse gas emissions as claimed in claim 8, wherein the reagent distributor is an annular reagent distributor (71); the annular reagent distributor (71) is disposed 20-100 cm below a liquid surface in the fermentation container (1); and a plurality of reagent nozzles (73) are defined on the annular reagent distributor (71) and configured to uniformly spray the reagents into the sludge.

10. The fermentation device for recovering the carboxylic acids while reducing the greenhouse gas emissions as claimed in claim 8, wherein the reagent regulation assembly (7) further comprises an inert gas storage chamber (76), an inert gas delivery pipe (77), and a plurality of inert gas blowing nozzles (78); the inert gas storage chamber (76) is disposed inside the reagent box (75) and is connected to the inert gas delivery pipe (77); an end portion of the inert gas delivery pipe (77) is horizontally disposed at a bottom of the fermentation container (1), and the plurality of inert gas blowing nozzles (78) are evenly distributed on the end portion of the inert gas delivery pipe (77).

11. The fermentation device for recovering the carboxylic acids while reducing the greenhouse gas emissions as claimed in claim 1, further comprising a venting assembly (8); wherein a venting port (103) is defined on a bottom of the fermentation container (1); the venting assembly (8) comprises a plurality of venting branch pipes (81), a venting intermediate box (82), and a venting pipe (83); an inlet of each of the plurality of venting branch pipes (81) faces downward, with a distance of 3-30 cm from a bottom surface of the fermentation container (1); the venting intermediate box (82) is a hollow box body, and a side wall of the venting intermediate box (82) is connected to the plurality of venting branch pipes (81); an end of the venting pipe (83) is connected to the venting intermediate box (82), and another end of the venting pipe (83) penetrates through fermentation container (1) through the venting port (103).

12. The fermentation device for recovering the carboxylic acids while reducing the greenhouse gas emissions as claimed in claim 1, wherein a plurality of instrument monitoring ports (112) are defined on the side wall of the fermentation container (1), and the plurality of instrument monitoring ports (112) are uniformly distributed along the side wall of the fermentation container (1) at a certain rotation angle from high to low; the monitoring instruments are embedded in the plurality of instrument monitoring ports (112), and the monitoring instruments comprises thermometers (91), pH meters (92), and water quality monitoring probes (93); the thermometers (91), the pH meters (92), and the water quality monitoring probes (93) are respectively used to continuously monitor the temperature, the pH value, and the water quality of the sludge inside the fermentation container (1); the controller (115) is configured to regulate a flow rate of the circulation pump (41) in the hydraulic circulation assembly (4) and a rotating speed of the agitator (61) in the agitator assembly (6) in real time, to thereby make data monitored by the thermometers (91) at different positions tend to be consistent, data monitored by the pH meters (92) at different positions tend to be consistent, and data monitored by the water quality monitoring probes (93) at different positions tend to be consistent.
